(19) United States
(12) Patent Application Publication
(10) Pub. No.: US 2022/0399093 A1
(43) Pub. Date: Dec. 15, 2022

(54) SYSTEMS AND METHODS OF MANAGING PRESCRIPTIONS

(71) Applicant: RxFlo, LLC, Salt Lake City, UT (US)

(72) Inventors: Jared Alan Stong, Draper, UT (US); Tyson W. Harper, Colorado Springs, CO (US); Benjamin Ray Clarke, North Salt Lake, UT (US)

(21) Appl. No.: 17/838,823

(22) Filed: Jun. 13, 2022

Related U.S. Application Data
(60) Provisional application No. 63/209,827, filed on Jun. 11, 2021.
(60) Provisional application No. 63/253,020, filed on Oct. 6, 2021.

(51) Int. Cl. G16H 20/10; G06F 3/0482

(52) U.S. Cl. CPC G16H 20/10; G06F 3/0482

(57) ABSTRACT

A system may generate a prescription file including a plurality of data fields, assign the prescription file to a first queue for processing, provide a first queue GUI that includes the plurality of data fields grouped in a plurality of information modules and indicates an approval status of each information module, and receive user input editing one or more data fields. The system may assign the prescription file to a second queue, provide a second queue GUI that includes the plurality of information modules and indicates the approval status of each information module, and receive user input setting the approval status of each information module. If an information module is not approved, the system may reassign the prescription file to the first queue. If all information modules are approved, the system may assign the prescription file to a third queue for further processing.

### RELATED APPLICATIONS

The present application claims priority under 35 U.S.C. § 119(e) to U.S. Provisional Patent Application No. 63/209,827, filed on Jun. 11, 2021, and U.S. Provisional Patent Application No. 63/253,020, filed on Oct. 6, 2021, each of which is incorporated herein by reference in its entirety.

### BACKGROUND INFORMATION

The provisioning (e.g., order, fulfillment, and delivery) of prescription medications to a patient, such as a patient under long-term care or hospice care, involves many different people in the patient's care team, such as a nurse, a nurse manager, a medical provider, a pharmacist, and a delivery agent. Members of the care team may use various different software applications to facilitate the provisioning of prescription medications. For example, a medical provider may use e-prescribe software to electronically submit a prescription to a pharmacy. A pharmacy may receive the e-prescription and use a separate on-site pharmacy management system to process and fill prescriptions, dispense medications, and manage inventory.

However, existing pharmacy management systems are outdated and inefficiently use computing resources. For example, existing pharmacy management systems may connect to multiple different remote third-party systems, such as the prescriber system, insurance providers, controlled substance databases, sister pharmacies, and the like, thereby using excessive computing resources to maintain such connections and slowing down the process of managing prescriptions and inventory. Conventional on-site pharmacy management systems also do not enable the use of mobile devices off-site.

Furthermore, the conventional methods of provisioning prescription medications to patients have various “black holes” that often cause delays in getting medications to the patients. These black holes may include, for example, illegible written prescriptions, illegible fax prescriptions, lost voicemail prescriptions, inaudible voicemail prescriptions, prescriptions still needing physician signatures, non-formulary medications awaiting approval, prescriptions with incorrect or missing information, lost prescriptions, prescriptions that have not yet been ordered by the medical provider, prescriptions that have not been received at the pharmacy, prescriptions that have not been processed at the pharmacy, wrong medications requested, and delayed or missed delivery, among other problems.

Delayed provisioning of prescription medications to a patient may result in increased suffering by the patient or early passing, emotional trauma to the patient's family, and problems for the care team, such as longer hours worked, burnout and frustration from broken processes, high employee (e.g., nurse) turnover, increased labor costs, inflated pharmacy costs, and declining quality scores.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative prescription management system.

FIG. 2 shows an illustrative environment in which the system of FIG. 1 may be implemented.

FIG. 3 shows an illustrative rendering of a graphical user interface (“GUI”) of an application that may be executed by a user device and through which a user may interact with the prescription management system of FG. 1 to process a prescription.

FIG. 4 shows an illustrative implementation of the GUI of FIG. 3 when the inbox queue is active.

FIG. 5 shows an illustrative implementation of the GUI of FIG. 3 when the entry queue is active.

FIGS. 6 and 7 show illustrative implementations of an action window of the GUI of FIG. 3 when the entry queue is active.

FIGS. 8 and 9 show illustrative implementations of the GUI of FIG. 3 when the review queue is active.

FIG. 10 shows an illustrative implementation of the GUI of FIG. 3 when the entry queue is active.

FIG. 11 shows an illustrative implementation of the GUI of FIG. 3 when the review queue is active.

FIG. 12 shows an illustrative implementation of the GUI of FIG. 3 when the claims queue is active.

FIG. 13 shows an illustrative implementation of an action window of the GUI of FIG. 3 when the claims queue is active.

FIG. 14 shows an illustrative implementation of the GUI of FIG. 3 when the pack queue is active.

FIG. 15 shows an illustrative implementation of the GUI of FIG. 3 when the final queue is active.

FIG. 16 shows an illustrative implementation of the GUI of FIG. 3 when the bag queue is active.

FIG. 17 shows an illustrative implementation of the GUI of FIG. 3 when the pick up queue is active.

FIG. 18 shows an illustrative method of reviewing and confirming information modules by way of various implementations of the GUI of FIG. 3.

FIG. 19 shows an illustrative architecture of a connected prescription platform.

FIG. 20 shows an illustrative method of real-time reporting of the status of prescription medication provisioning.

FIG. 21 shows another illustrative method of real-time reporting of the status of prescription medication provisioning.

FIG. 22 shows an illustrative dashboard GUI of a visibility system that may be presented by way of a user device.

FIG. 23 shows an illustrative GUI that shows a status of a selected prescription order.

FIG. 24 shows an illustrative detailed status report of a prescription.

FIG. 25 shows another illustrative GUI that may be presented by a user device.

FIG. 26 illustrates an exemplary computing device that may be specifically configured to perform one or more of the processes described herein.

### DETAILED DESCRIPTION

Prescription management systems and methods will be described herein with reference to the drawings. A connected prescription platform and system architecture for prescription medication provisioning will also be described herein with reference to the drawings.

The prescription management systems described herein provide a fully end-to-end paperless prescription processing ecosystem, from receipt of the prescription from the prescriber to pick up or delivery of the prescribed medication to the patient. The prescription management system also enables processing of prescriptions from remote locations (e.g., locations other than at the physical pharmacy). The connected prescription platforms described herein track the status of prescription orders as prescriptions are processed across different devices, systems, and by different care team personnel. The connected prescription platforms provide any user at any connected device with real-time on-demand visibility to information about the status of prescription processing and prescription medication provisioning. Thus, the connected prescription platforms automatically provide end-to-end visibility, from the prescriber to the pharmacy to delivery to the patient. This visibility provides accountability for the care team (nurse, physician/prescriber, pharmacy) and eliminates black holes associated with provisioning prescription medication to patients. These and other benefits and advantages of the prescription management systems and connected prescription platforms will be described in more detail in the discussion that follows.

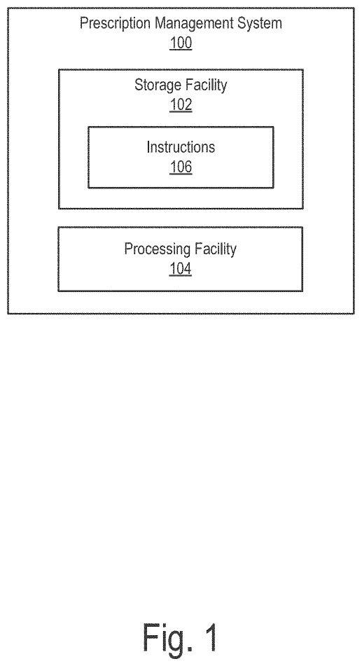

FIG. 1 shows an illustrative prescription management system 100 (“system 100”). System 100 may be included in, implemented by, or connected to one or more computing systems or computing devices. As shown in FIG. 1, system 100 includes, without limitation, a memory 102 and a processing unit 104 selectively and communicatively coupled to one another. Memory 102 and processing unit 104 may each include or be implemented by hardware and/or software components (e.g., processors, memories, communication interfaces, instructions stored in memory for execution by the processors, etc.). In some examples, memory 102 and processing unit 104 may be distributed among multiple devices and/or multiple locations as may serve a particular implementation.

Memory 102 may maintain (e.g., store) executable data used by processing unit 104 to perform any of the operations described herein. For example, memory 102 may store instructions 106 that may be executed by processing unit 104 to perform any of the operations described herein. Instructions 106 may be implemented by any suitable application, software, code, and/or other executable data instance. Memory 102 may also maintain any data received, generated, managed, used, and/or transmitted by processing unit 104. For example, memory 102 may maintain prescription data, patient data, user data, drug data, document data, insurance data, and/or any other suitable data.

Processing unit 104 may be configured to perform (e.g., execute instructions 106 stored in memory 102 to perform) various operations associated with managing a prescription. Operations that may be performed by processing unit 104 are described herein. In the description that follows, any references to operations performed by system 100 may be understood to be performed by processing unit 104 of system 100.

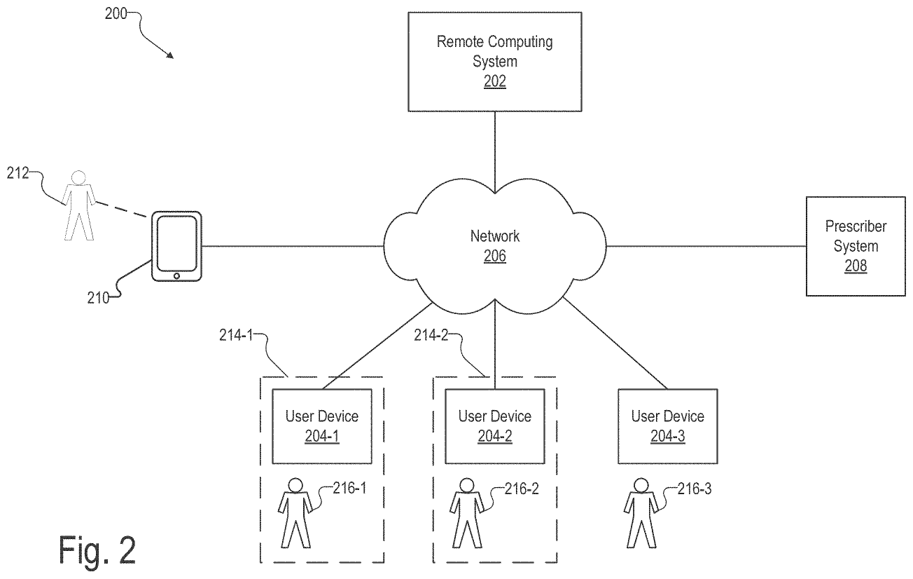

FIG. 2 shows an illustrative environment 200 in which system 100 may be implemented. As shown, a remote computing system 202 is communicatively coupled to user devices 204-1 to 204-3 (collectively, “user devices 204”) by way of a network 206. While FIG. 2 shows that remote computing system 202 is connected to three user devices 204, remote computing system 202 may be coupled to any other number of user devices 204 (e.g., one, two, or more than three) as may serve a particular implementation.

Remote computing system 202 may include one or more computing devices (e.g., servers) configured to perform any of the operations described herein. Examples of a user device 204 may include, but are not limited to, a wireless computing device, a personal computer, a desktop computer, a mobile computing device (e.g., a mobile phone device, a tablet computer, a laptop computer, etc.), a personal digital assistant, and/or any other computing device that may perform one or more of the operations described herein.

Network 206 may be a local area network, a wireless network (e.g., Wi-Fi), a wide area network, the Internet, a cellular data network, and/or any other suitable network. Data may flow between components connected to network 206 using any communication technologies, devices, media, and protocols as may serve a particular implementation.

In some examples, as shown in FIG. 2, a prescriber system 208 may also connect with remote computing system 202 and/or user devices 204 by way of network 206 (and/or by way of another network). Prescriber system 208 may be implemented by one or more computing devices, and may be used by a prescriber (e.g., a physician) to maintain and manage patient information, generate and electronically transmit prescriptions (e.g., to remote computing system 202), and/or perform any other suitable operations. Prescriber system 208 may be associated with a medical facility, such as a hospital, a surgical facility, a medical clinic, a doctor's office, a dentist's office, a nursing home, a hospice facility, a rehab facility, an assisted living facility, or any other similar medical or healthcare facility. Prescriber system 208 may include one or more computing devices and may be provided locally at the prescriber's physical location and/or may be distributed across multiple locations and/or based in the cloud.

As also shown in FIG. 2, a patient device 210 associated with a patient 212 may connect with remote computing system 202, user devices 204, and/or prescriber system 208 by way of network 206 (and/or another network). Patient device 210 may be any suitable computing device, such as a mobile device (e.g., a smartphone or a tablet computer), a personal computer (e.g., a desktop computer, a laptop computer), or a wearable device (e.g., a smartwatch).

User devices 204, prescriber system 208, and/or patient device 210 may each store and execute an application that provides a user 216 and/or patient 212 with access to information and data maintained by system 100. The application may be any type of application as may suit a particular implementation. For example, the application may be a client-side application (e.g., a “thin client application” such as a mobile application) locally installed on user devices 204, prescriber system 208, and/or patient device 210 and communicatively coupled with system 100 (e.g., with an application and data stored on remote computing system 202). As another example, the application may be a web application accessible through a browser application installed on user devices 204, prescriber system 208, and/or patient device 210. As will be explained below in more detail, system 100 facilitates management of prescriptions to be filled and dispensed at pharmacy locations 214.

One or more user devices 204 may be associated with a distinct physical pharmacy location 214 (e.g., pharmacy location 214-1or pharmacy location 214-2), which is indicated by a dashed-line box. A pharmacy location 214 may be any physical location where medications are stored and/or dispensed, such as a retail pharmacy, a mail-order or closed-door pharmacy, a warehouse, or a mobile pharmacy (e.g., a delivery van). A user device 204 may be associated with a pharmacy location 214 in any suitable way. For example, a user device 204 (e.g., user devices 204-1 and 204-2) may be physically located at a pharmacy location 214, used by a user 216 (e.g., a user 216-1 or 216-2) while the user 216 is physically located ata pharmacy location 214, used by an employee or other person given access rights by an operator of pharmacy location 214, and/or maintain and/or provide access to data (e.g., patient or customer data, inventory data, etc.) associated with a pharmacy location 214. As shown in FIG. 2, user devices 204-1 and 204-2 are physically located on the premises of pharmacy locations 214-1 and 214-2, respectively, while user device 204-3 is not physically located on the premises of any pharmacy location 214. For example, user device 204-3 may be located remotely from pharmacy location 214-1 and/or 214-2. Nevertheless, user device 204-3 may be associated with pharmacy location 214-1 and/or 214-2. For example, user 216-3 may be a remote data entry clerk working from home that uses user device 204-3 (e.g., a tablet computer) to input prescription information to system 100. Alternatively, user 216-3 may be a delivery driver that uses user device 204-3 to view delivery routes and schedules to physically deliver medications to patients (e.g., to patient 212).

While FIG. 2 shows that each pharmacy location 214 is associated with a single user device 204, each pharmacy location 214 may be associated with any other number of user devices 204. Moreover, while FIG. 2 shows that one user 216 (e.g., a pharmacist, a pharmacy technician, a data entry clerk, etc.) is located at each pharmacy location 214, any other number of users 216 may be located at each pharmacy location 214. Similarly, while FIG. 2 shows only one remote user device 204-3 is associated with pharmacy location 214-1 and/or pharmacy location 214-2, any other number of remote user devices may be associated with pharmacy location 214-1 and/or pharmacy location 214-2.

In some examples, multiple pharmacy locations 214 may be associated with (e.g., owned by, operated by, managed by, share inventory with, have access to the same patient or customer data, etc.) the same pharmacy. For example, pharmacy location 214-1 may be a first retail location (e.g., in City A) of a pharmacy and pharmacy location 214-2 may be a second retail location (e.g., in City B) of the pharmacy.

Although not shown in FIG. 2, other systems may be connected to remote computing system 202, user devices 204, and/or prescriber system 208 by way of network 206, such as third-party switches (e.g., RelayHealth Pharmacy Solutions or eRx Network), a controlled substance database, and a National Drug Code (NDC) database.

System 100 facilitates management, by way of one or more applications executed by one or more user devices 204, of prescription fulfillment, from prescription receipt to dispensing and delivery of the prescribed medication. System 100 may also facilitate confirmation of delivery of the prescribed medication. In some examples, system 100 is implemented entirely by remote computing system 202. In alternative examples, system 100 is distributed across any two or more of remote computing system 202, user device(s) 204, prescriber system 208, and patient device 210. Illustrative operations that may be performed by system 100 will now be described.

System 100 may receive and store a prescription. As used herein, a prescription may refer to an instruction from a prescriber that authorizes a specific drug to be dispensed to a specific patient. The prescription may be in any format, such as physical (e.g., a paper document), digital (e.g., an e-prescription, an image file, an email, a data file, etc.), or audio (e.g., a voice message). System 100 may receive the prescription in any suitable way, such as by email, fax, text message, SMS message, phone call, voice message, or any other suitable means. In some examples, a pharmacy may be assigned a unique address (e.g., email address, fax number, phone number, short code, etc.), and system 100 may automatically receive and store prescriptions that are sent to the unique address. In some examples, a physical prescription received by a pharmacy may be scanned or photographed, such as by user device 204 (e.g., a smartphone camera) or a scanning device coupled to user device 204, and a scanned image of the prescription may be stored by system 100 (e.g., in memory 102). In yet further examples, system 100 may access the prescription from a remote computing system (e.g., prescriber system 208), such as by scanning a bar code, QR code, or other code representative of the prescription. In some examples, a user may manually upload a prescription (e.g., a voice message audio file, a scanned image, an email, etc.).

Once system 100 has received and stored the prescription, a user may use the stored prescription to generate a prescription file. A prescription file contains various fields of information from the prescription and other records (e.g., patient data, prescriber data, drug data, etc.) that may be used by a pharmacist to dispense and deliver the prescribed medication to the patient. As will be explained below in more detail, a prescription file may include information such as patient information (e.g., name, date of birth, age, gender, address, etc.), drug information (e.g., drug name, dosage, drug quantity), number of refills, label information, insurance information, prescriber information, instructions, and the like. System 100 may mark a prescription file as “open” until the medication has been confirmed to have been picked up or delivered, at which time system 100 marks the prescription file as “closed.” Although closed prescription files are not assigned to a queue for processing, they may be stored by system 100 and accessed and viewed by a user.

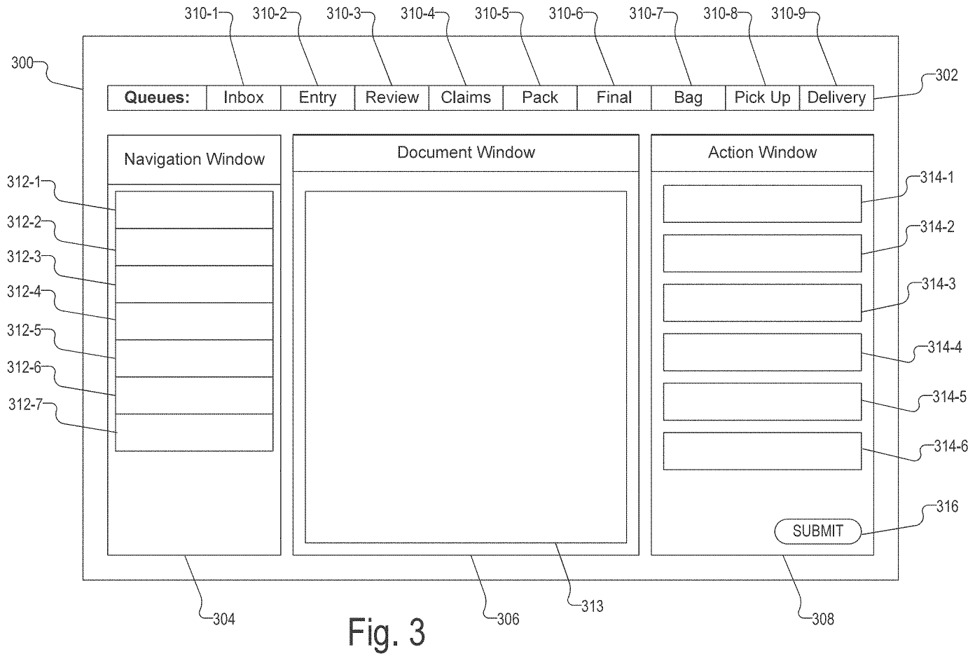

FIG. 3 shows an illustrative rendering of a graphical user interface (“GUI”) 300 of an application that may be executed by a user device 204 and through which a user 216 may interact with system 100 to process a prescription. As shown, GUI 300 includes a queue menu bar 302, a navigation window 304, a document window 306, and an action window 308. GUI 300 may include any additional or alternative windows and/or graphical elements as may serve a particular implementation, including a settings menu, user account information, inventory information, and the like.

System 100 may assign documents or files received by system 100 (e.g., emails, faxes, uploaded documents, voice messages, etc.) to the inbox queue and may assign each open prescription file to one of various different prescription file queues, depending on the stage of processing the prescription file. For example, each prescription file may include a queue field indicating the currently assigned queue for that prescription file. Thus, the inbox queue is assigned a set of one or more received documents to be processed, and each prescription file queue is assigned a set of one or more open prescription files to be processed by a user (e.g., input information, edit, review, approve, print, etc.) prior to dispensing or delivering medication associated with the prescription file.

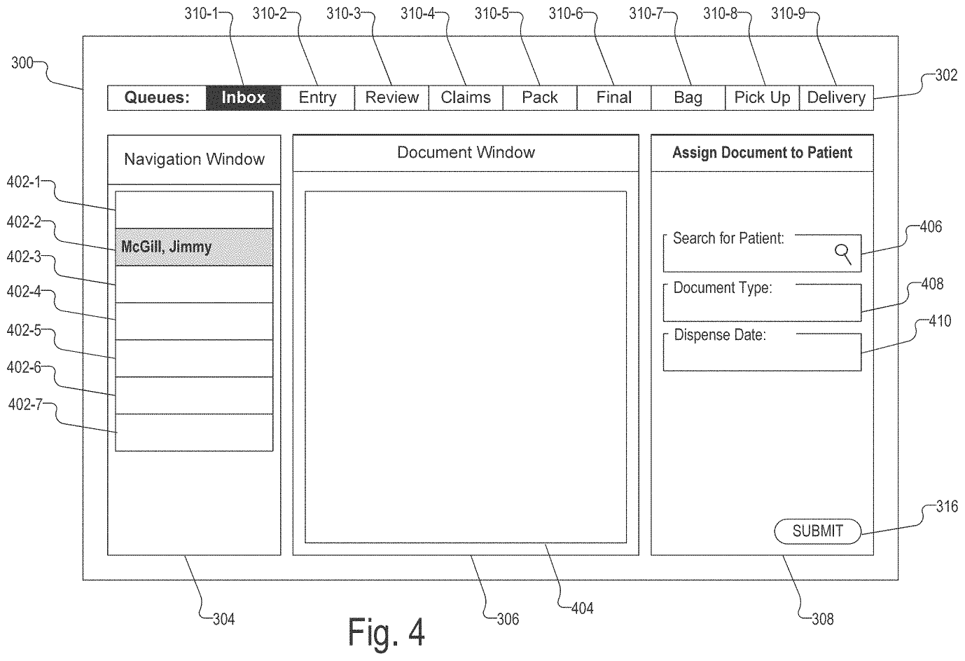

Queue menu bar 302 provides a number of selectable queue buttons 310 for activating a particular queue, including an inbox queue button 310-1 for activating an inbox queue, an entry queue button 310-2 for activating an entry queue, a review queue button 310-3 for activating a review queue, a claims queue button 310-4 for activating an insurance claims queue, a pack queue button 310-5 for activating a pack queue, a final queue button 310-6 for activating a final review queue, a bag queue button 310-7 for activating a bag queue, a pick up queue button 310-8 for activating a pick up queue, and a delivery queue button 310-9 for activating a delivery queue. Any of these queues may be combined into a single queue as may serve a particular implementation, and queue menu bar 302 may correspondingly include fewer or more or alternative queue buttons 310 as may serve a particular implementation. Selecting a particular queue button 310 to activate the queue enables a user to view and interact with documents or prescription files that are currently assigned to the active queue. In some examples, queue menu bar 302 and/or navigation window 304 may indicate a total number of prescription files that are currently assigned to that queue. For example, as shown in FIG. 4 each button in queue menu bar displays the number of prescription files in the respective queue.

Navigation window 304 includes a plurality of selectable identifiers 312 (e.g., identifiers 312-1 to 312-7) that each identify a distinct document (when the inbox queue is active) or a distinct prescription file (when any of the other queues are active).

Navigation window 304 allows a user to navigate among available documents or prescription files within the active queue by selecting a particular identifier 312. Navigation window 304 may also include options to sort and/or filter identifiers 312 and or search for a particular identifier 312 or group of identifiers 312. In some examples, identifiers 312 in navigation window 304 may be toggled between an expanded view or a condensed view. In the condensed view, identifiers 312 provide only a brief identification (e.g., ID number or patient name) of the associated document or file, while in the expanded view identifiers 312 provide additional information or details of the associated document or file.

Upon selection of a particular identifier 312 within navigation window 304, document window 306 shows the document(s) 313 and/or other information that is/are associated with the selected identifier. In some examples, document window 306 also includes various graphical elements (not shown), such as buttons, menus, and selectable options, that may allow a user to perform an action with respect to document 313 or information presented within document window 306. For example, document window 306 may include one or more selectable options to delete a document (e.g., spam documents), annotate a document, adjust image settings (brightness, contrast, etc.) to facilitate reading of the document, adjust a zoom level, change pages (on a multi-page document), redact information, print, download, rotate, and/or perform any other suitable document action.

Action window 308 provides various graphical elements 314 (e.g., information modules, text entry fields, etc.) that allow a user to take some queue-specific action with regard to the selected identifier (document or prescription file), such as entering data, editing or modifying data, and approving or rejecting data. Action window 308 may also include selectable button 316 to submit any changes or edits made by way of action window 308.

The queue button 310 for the active queue and the identifier 312 for the active document or active prescription file may be visually indicated to the user in queue menu bar 302 and/or navigation window 304, respectively, such as by highlighting, changing a size/shape of text or graphical elements, adding a graphical element (e.g., an arrow), changing a color, or any other suitable graphical indicator.

The particular layout of GUI 300 shown in FIG. 3 is not limiting, as any other design or layout may be used. For example, navigation window 304, document window 306, and action window 308 may have any other suitable positioning. For instance, action window 308 may be positioned beneath navigation window 304 and document window 306. In some examples, any one of windows 304, 306, and 308 may be minimized, resized, or repositioned as desired by the user (e.g., by selectable resizing handles). Moreover, GUI 300 may have a unique layout for a particular queue that is different from the layout of GUI for other queues. In some examples, document window 306 and action window 308 may be combined into a single window.

System 100 may generate a prescription file and add prescription information to the prescription file in any suitable way. For example, when the inbox queue is active, navigation window 304 may present a list of document identifiers representing documents that have been received and are currently stored by system 100. In some examples, navigation window 304 provides a listing of only the documents that have not yet been associated with a prescription file.

FIG. 4 shows an illustrative implementation of GUI 300 when the inbox queue is active. As shown, navigation window 304 includes a plurality of document identifiers 402 (e.g., document identifiers 402-1 to 402-7) each representing a received document (e.g., a prescription) to be processed. Document window 306 shows a prescription document 404 associated with document identifier 402-1, which has been selected in navigation window 304. Action window 308 includes a patient name field 406 by which a user may input, search, and/or select a patient name to associate with the document. Action window 308 also includes a document type field 408 for indicating a type of the document presented in document window 306. If the document is a new prescription, the user may select “Rx Order” as the document type to create a new prescription file. If the document is not a new prescription (e.g., a medical chart, patient record, other patient information, insurance information, etc.), the user may select a different option to add the document to an existing patient profile for the patient. Action window 308 may optionally also include other input fields, such as a dispense date, a priority level, a dispense method (e.g., pick-up or delivery), and/or any other information. Action window 308 may also include a selectable button which, upon selection, associates document 404 shown in document window 306 with the patient profile for the patient selected in action window 308. When the user selects submit button 316, if the user selected “Rx Order,” system 100 creates and stores a new prescription file and associates the prescription file with the patient. In some examples, the user may also create a new patient profile in action window 308 if the patient listed on the document is a new patient or not currently stored in system 100.

Upon creating a new prescription file, system 100 may stay in the inbox queue (keep the inbox queue as the active queue) for processing another document in the inbox (“queue mode”), or system 100 may activate the next queue (e.g., the entry queue) and select a prescription file to process (“follow mode”).

In queue mode, system 100 may automatically select the next document identifier 402 in navigation window 304 (e.g., identifier 402-3) (or prescription file identifier in a subsequent queue), or the user may manually select, in navigation window 304, a document identifier 402 (or prescription file identifier) to process next.

In follow mode, system 100 automatically follows a particular prescription file through all queues by automatically advancing to each next queue upon completion of each queue to continue processing the same prescription file through all queues. In each next queue, system 100 may visually indicate (e.g., highlight), in navigation window 304, the prescription file currently in follow mode for easy selection by the user. In some examples, system 100 may make the prescription file the active prescription file upon advancing to the next queue.

Queue mode or follow mode may be set as a default mode for processing, and the default mode may be selected or changed by a user. For example, in one or more queues, GUI 100 may include (e.g., in action window 308) a selectable option for a user to select follow mode. Upon selection of follow mode, system 100 operates in follow mode and automatically activates the next queue upon completion of the queue (e.g., submission of all required information in action window 308).

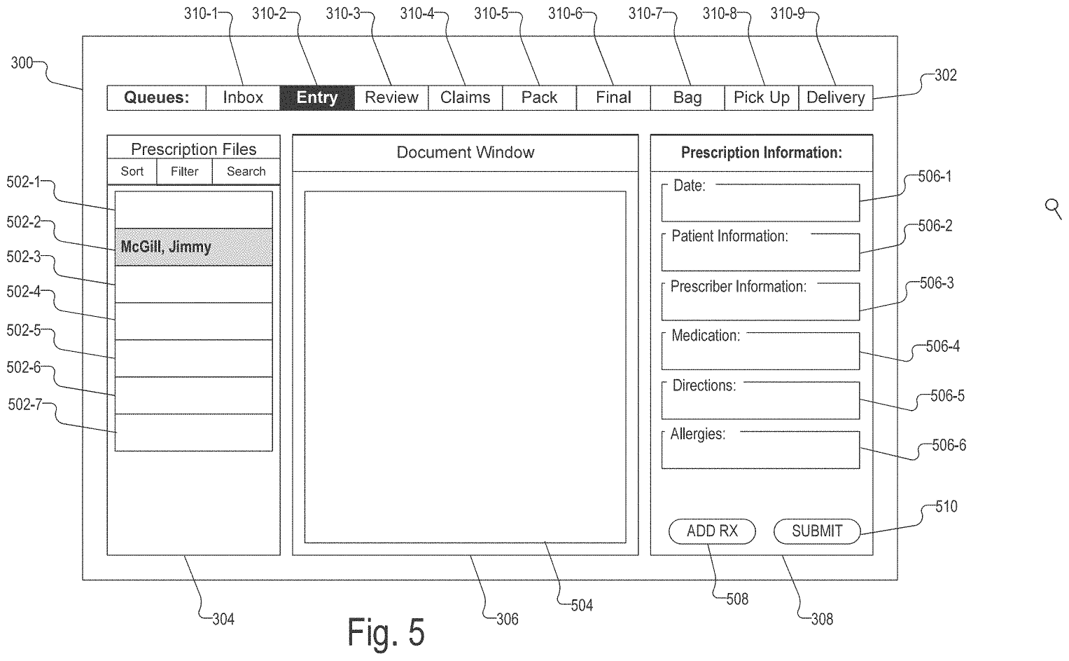

When the entry queue is active, a user may use the document displayed in document window 306 to enter, in action window 308, information into data fields of the prescription file. FIG. 5 shows an illustrative implementation of GUI 300 when the entry queue is active. As shown, navigation window 304 includes a plurality of prescription file identifiers 502 (e.g., prescription file identifiers 502-1 to 502-7) each representing a unique prescription file previously generated in the inbox queue and that are ready for entry of certain required information. Prescription file identifiers 502 may be arranged in any suitable order. For example, prescription file identifiers 502 may be arranged alphabetically by patient name, by a file number, or by a date/time created. Document window 306 shows the prescription document 504 associated with the selected prescription file, and action window 308 allows the user to input information for the selected prescription file.

In some examples, the data fields may be grouped into different information modules 506 (groups of related or associated information) such as for billing/insurance information, prescriber information, patient diagnosis, prescribed drug, directions for taking the drug, quantities of the drug, dispense code, dosage information, number of refills, and the like. As shown in FIG. 5, action window 308 includes a prescription date module 506-1, a patient information module 506-2, a prescriber information module 506-3, a medication module 506-4, a directions module 506-5, an allergies module 506-6. Although not shown in FIG. 5, action window may also include a dispense date module, a dosage module, a warnings module, and a clinical review module. In some examples, a user may scroll through action window 308 to view all information modules 506. Action window 308 may include any additional and/or alternative information modules 506 as may suit a particular implementation.

Action window 308 (e.g., information modules 506) may also show information already associated with the prescription file or already stored or known by system 100, such as the patient's name (which may be obtained from the patient profile), prescriber information (e.g., prescriber contact information), prestored directions, drug information such as National Drug Code (NDC) number (stored by system 100 or accessed from a third-party database), etc.

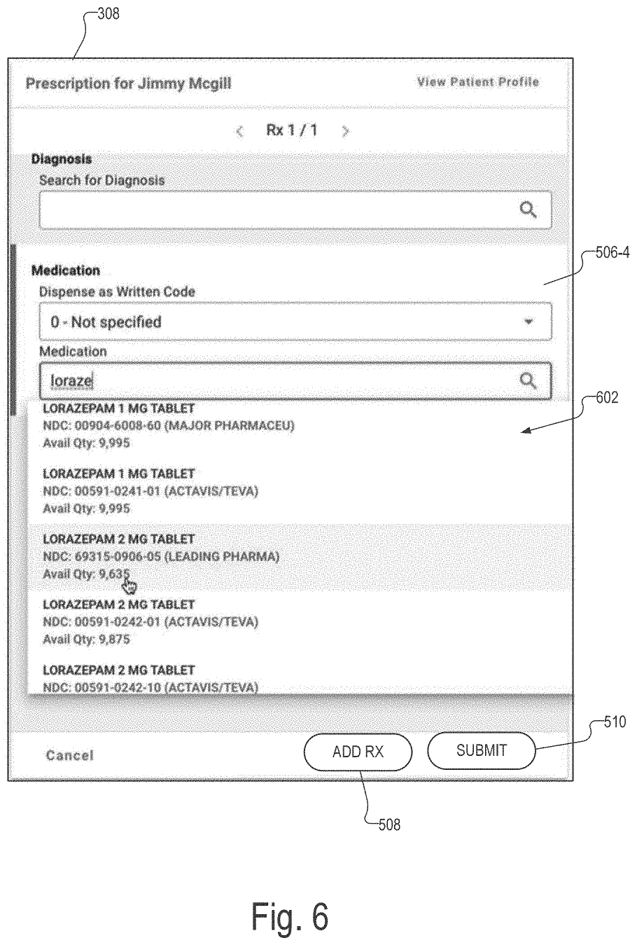

In some examples, when entering the medication indicated by the prescription, system 100 may show the user the available quantity of the medication that has been selected. For example, FIG. 6 shows an illustrative implementation of action window 308 when the entry queue is active. As shown, each drug presented in a drop-down menu 602 in medication information module 506-4 includes the drug's NDC number and the available quantity. The available quantity refers to the quantity of pills, tablets, capsules, etc. of that drug that are currently in inventory for the pharmacy and have not been committed to other patients. In this way, the user can easily see if there are enough drugs to fill the current prescription. The available quantity and inventory information may be specific to the particular pharmacy location or may be based on multiple locations associated with a pharmacy entity. In this way, the user can easily see if other pharmacy locations within the organization may have extra supply of drugs that are currently needed by the pharmacy location. When drugs are committed to a patient, dispensed, or delivered, as explained below, system 100 may update the inventory information for that drug. System 100 may assume that drugs are committed at any suitable point in the processing of prescriptions. For example, system 100 may commit drugs upon completion of the entry queue. Alternatively, system 100 may commit drugs upon dispensing the drugs (e.g., upon completion of the pack queue), as will be explained below.

Referring again to FIG. 5, the user may use directions module 506-5 of action window 308 while the entry queue is active to enter directions for taking the medication, which directions may be printed when the drug is dispensed and delivered to the patient. In some examples, the user may manually type out the directions. Alternatively, the directions may be pre-stored and this field may be automatically populated when the particular drug (and optionally dosage information) is entered by the user. However, in some situations the user device 204 that may be used to enter the prescription file information is a mobile device, such as a mobile phone or a tablet computer. As a result, it may be difficult for the user to manually type out the full directions. Accordingly, system 100 may store short codes that a user may enter to provide the directions information. For example, upon entering the short code “1 bid” system 100 may populate a directions field within directions module 506-5 field with “TAKE 1 TABLET(S) BY MOUTH TWICE DAILY.”

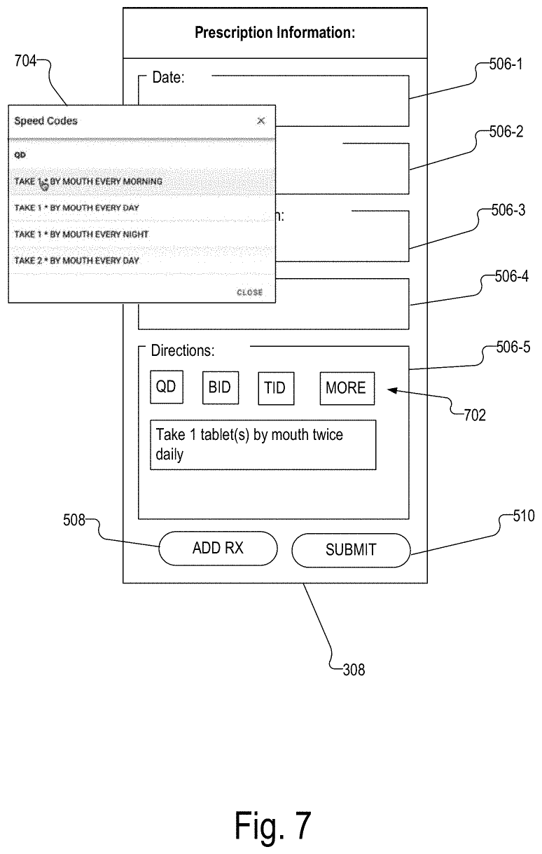

In other examples, action window 308 may include short code selectable buttons that may be selected to populate the directions field, as illustrated in FIG. 7. FIG. 7 shows an illustrative implementation action window 308 when the entry queue is active. As shown in FIG. 7, directions module 506-5 includes different short code buttons 702 (“OD”, “BID”, “TID”, and “More” for more options), each of which may include a submenu of possible pre-stored directions that the user may select. For example, a user may select the “QD” button 702-1, an in response system 100 may present a popup window 704 that allows the user to select the appropriate directions information. System 100 then populates directions module 506-5 with the selected directions information. In this way, a user can easily process the prescription file by way of a touchscreen mobile device (e.g., a smartphone or tablet computer). In some examples, a user may create a custom short code (and also a short code button), such as by selecting an “ADD SIG” button (not shown) within directions module 506-5 and entering the short code and the directions associated with the short code. Thus, the user can create short codes and short code buttons on the fly.

Referring again to FIG. 5, the user may also enter, by way of various information modules 506 within action window 308, dispense date, priority information, and notes (e.g., patient notes, prescription notes, etc.). The priority information may be used to indicate a priority of the prescription file. For example, the user may indicate a specific time when the medication needs to be picked up or delivered, a priority level (e.g., on a range of 1 to 5 with 1 being most urgent), an estimated time when the medication is needed, or simply “stat” or “immediate.” In some examples, system 100 may arrange prescription file identifiers 502 in navigation window 304 in each of the queues based on their assigned priorities, with the highest priority at the top. In this way, a user may quickly and easily process the most urgent prescription files first. In some examples, system 100 may additionally or alternatively flag or otherwise visually indicate high priority prescription files in navigation window 304.

In some situations, a single prescription document may contain information for multiple different drugs prescribed to a patient. This information may be contained on a single page or may be on multiple different pages (such as when system 100 receives a multiple-page fax). Accordingly, the user may select an add Rx button 508 in action window 308, as shown in FIGS. 5 and 6, to split the prescription file into multiple prescription files. Upon selecting add Rx button 508, system 100 may create a new prescription file, which may then appear in the list of prescription file identifiers 502 in navigation window 304. In some examples in which the information for the new prescription file is on its own page of the document, selecting the add Rx button 508 may cause the currently viewed page of the document shown in document window 306 to be automatically separated from the document file (e.g., printed or saved separately) and associated only with the new prescription file. Alternatively, the entire multi-page document may be associated with the new prescription file. The user may then annotate the document, as explained above, to indicate which page is associated with which prescription file. In some examples, after selecting add Rx button 508, system 100 may prompt the user to select whether to associate the entire multi-page document with the new prescription file or to associate only the currently viewed page with the new prescription file. The user may also designate the user's preference as a default setting.

When a prescription file is split to create one or more additional prescription files and while processing prescription files in the follow mode, system 100 may continue operating in the follow mode in various different ways. In some examples, system 100 may continue to follow the original prescription file and then, after completing processing of the original prescription file (e.g., after completing the final queue or some other designated queue), system 100 may return to the entry queue to finish processing the new prescription file. Alternatively, system 100 may process both prescription files in each queue (similar to the queue mode but only for the related prescription files) before advancing to the next queue. In some examples, system 100 may prompt the user to indicate how the user would like to proceed with the follow-mode (e.g., which prescription file to follow first or to follow both simultaneously, or to switch out of the follow mode).

After the user has finished filling out the fields in action window 308 of the entry queue, the user may select submit button 510 to complete the entry queue and advance the prescription file to the next queue for further processing.

While the review queue is active, a pharmacist or other authorized user may review and confirm the accuracy of the prescription file information that has been entered, such as by cross-checking the entered information with the information shown in the associated prescription document. The user may also perform any other reviews, such as clinical reviews. The review of the prescription file information may, in some examples, be delayed to a later queue (e.g., the final queue), as explained further below.

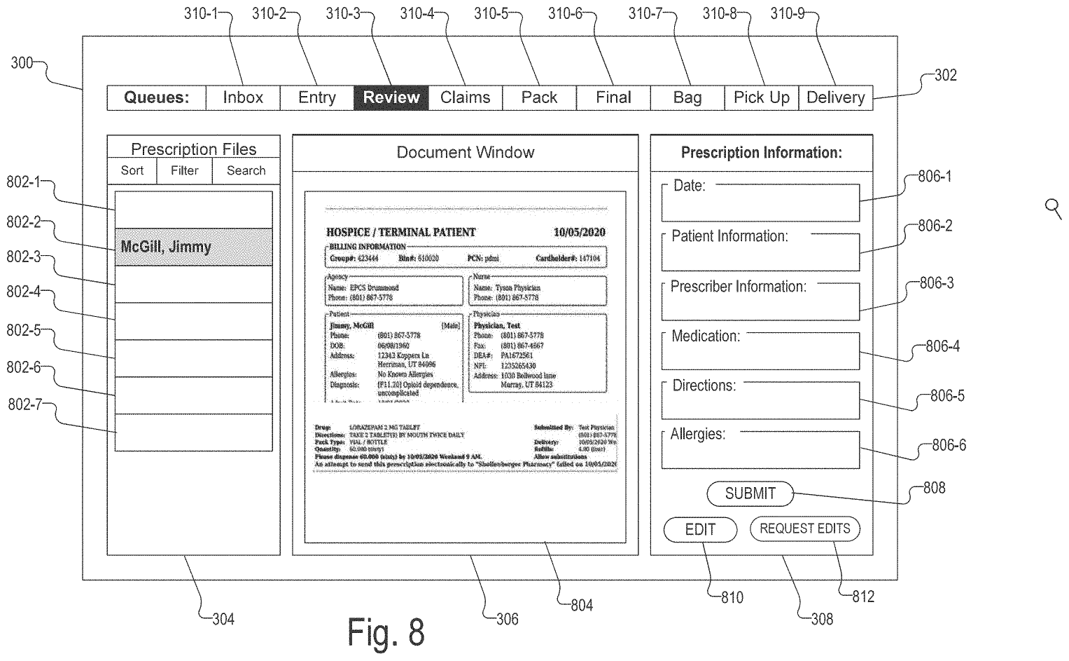

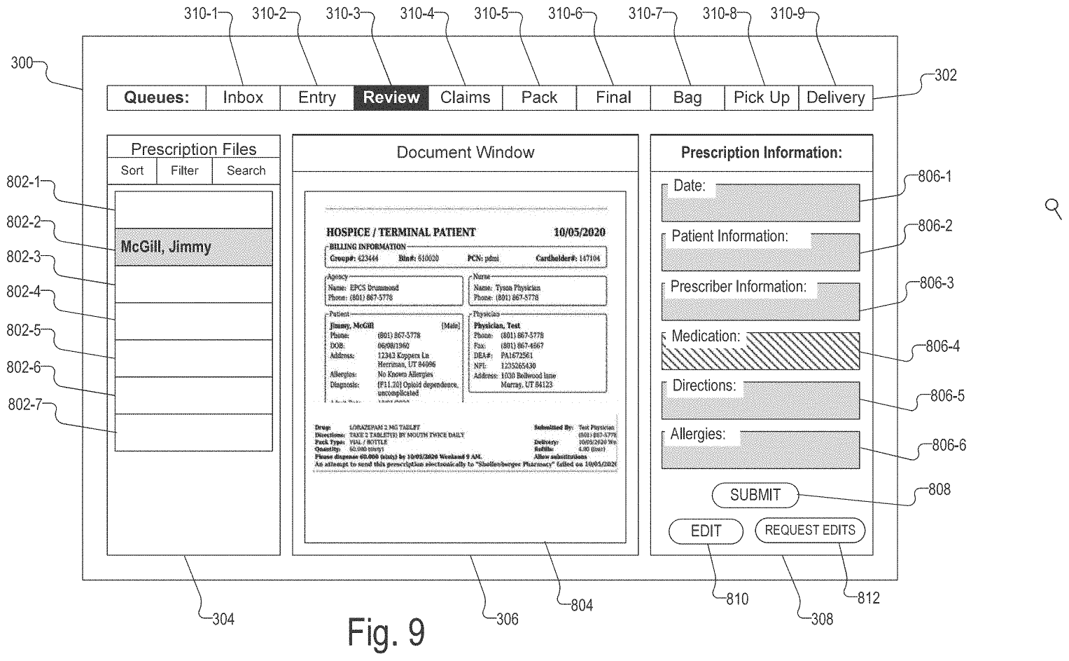

FIGS. 8 and 9 show illustrative implementations of GUI 300 when the review queue is active. As shown, navigation window 304 includes a plurality of prescription file identifiers 802 (e.g., prescription file identifiers 802-1 to 802-7) each representing a unique prescription file that is ready for review and approval of the information entered in the entry queue. In the review queue, a document 804 associated with the selected prescription file identifier 802 is displayed in document window 306 and the prescription file information is shown in various information modules 806 in action window 308. Each information module 806 is a distinct graphical element that displays the information for each field (or group of associated fields) of the prescription file. In some examples, information modules 806 of the review queue are the same as information modules 506 of the entry queue. For example, FIG. 8 shows that action window 308 of the review queue includes a prescription date module 806-1, a patient information module 806-2, a prescriber information module 806-3, a medication module 806-4, a directions module 806-5, and an allergies module 806-6. Although not shown, action window 308 may include other information modules, such as a dispense date module, a dosage module, a warnings module, and a clinical review module. Any additional and/or alternative information modules may be included in action window 308.

In some examples, system 100 does not move the prescription file to the claims queue until a user has reviewed and confirmed or approved each information module 806. A user may provide input to set or change the approval status of each information module 806, and system 100 may visually indicate the approval status of each information module 806. In some examples, the approval status may be “Needs Review,” “Approved,” or “Not Approved.” The approval status may be visually indicated in any suitable way, such as by a flag or other graphical icon or element on the information module 806, changing the background color of the information module 806, or any other suitable graphical indication. In some examples, the status of each information module may be indicated by a background color of the information module 806. For example, white (represented by a white background in FIGS. 8 and 9) indicates “Needs Review,” green (represented by a light gray background in FIG. 9) indicates “Approved,” and red (represented by cross-hatching in FIG. 9) indicates “Not Approved.”

The user may change the approval status of an information module 806 in any suitable way. In some examples, a user may interact with the information module 806, such as by selecting a selectable button or other element on the information module 806, checking a checkbox in the information module 806, pressing a hotkey associated with each information module 806 (e.g., Ctrl+M for the medication module), selecting the information module 806 (e.g., with a mouse and pointer, a finger, or a stylus anywhere on the information module 806), or performing a predefined gesture with respect to the information module 806. For example, the approval status may change to the next approval status in a repeating cycle of approval statuses in response to each selection of the information module 806 (e.g., first “Needs Review,” then “Approved,” then “Not Approved,” then back to “Needs Review,” and so on).

Additionally or alternatively, a first predefined user input may indicate a first approval state (e.g., a single-selection action such as a single-tap or single-click may set the status to “Approved”), a second predefined user input that is different from the first predefined user input may indicate a second approval state (e.g., a double-selection such as a double-tap or double-click) may set the status to “Not Approved”), and a third predefined user input that is different from the first predefined user input and second predefined user input may indicate a third approval state (e.g., a long-press may set the approval status to “Needs Review”). As another example, a first gesture (e.g., a swipe right or a single-finger swipe) may change the status to “Approved,” a second gesture (e.g., a swipe left or a double-finger swipe) may change the status to “Not Approved,” and a third gesture (e.g., a swipe up and/or down or a three-finger swipe) may change the status to “Needs Review.” As yet another example, a user may select a particular information module 806 (e.g., by a tap or click on the information module 806) to activate that information module 806 and then select an appropriate status from a menu of approval statuses. As shown in FIG. 9, information modules 806 that have been approved have a green background color (indicated by gray shading) and the medication module 806-4 has not been approved and thus is presented with a red background (indicated by cross-hatching).

Action window 308 includes a submit button 808 that may be selected by a user to submit the review and move the prescription file to the appropriate queue, which system 100 determines based on the approval status of information modules 806. When all information modules 806 have been approved, system 100 advances the prescription file to the next queue (the claims queue). If, however, any of the information modules 806 have a “Not Approved” status, system 100 moves the prescription file back to the entry queue to enable a user to edit the prescription file.

In some examples, submit button 808 may be selected only when all information modules 806 have an “Approved” status. If any information module 806 has a “Not Approved” status, submit button 806 is not selectable and a selectable edit button 810 may be selected to return the prescription file to the entry queue and activate the entry queue so that the user may promptly make the necessary changes to the information module 806. Alternatively, the user may select a request edit button 812 (e.g., “Request Edits”) to move the prescription file to the entry queue but keep the review queue active. Thus, the user may continue working in the review queue while another user (or the same user at a later time) may make the needed corrections to the information module 806 in the entry queue.

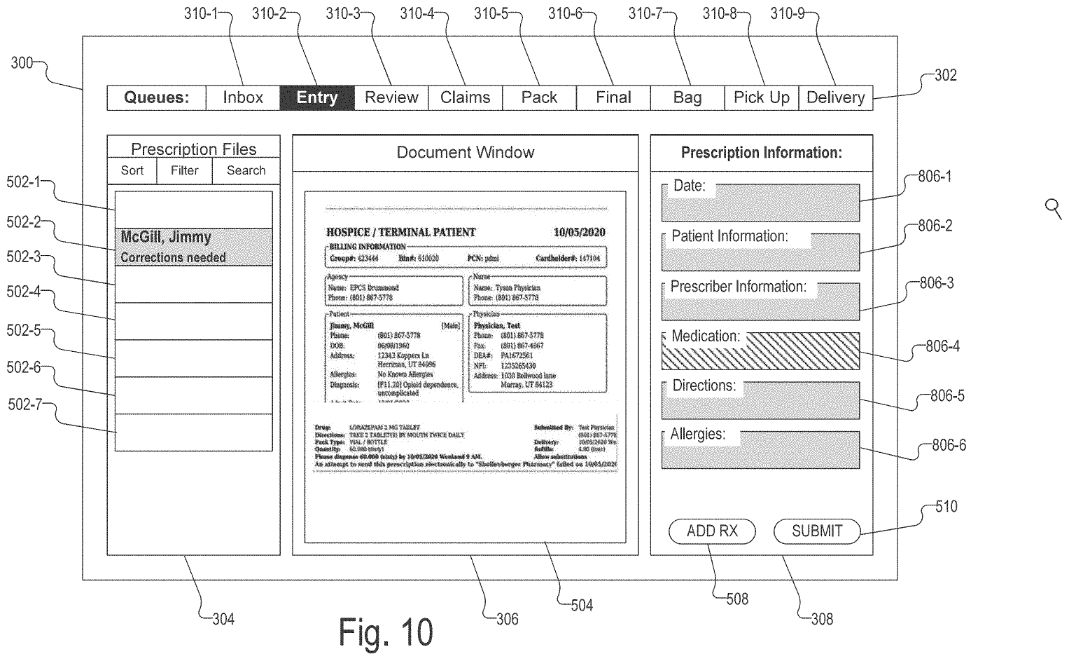

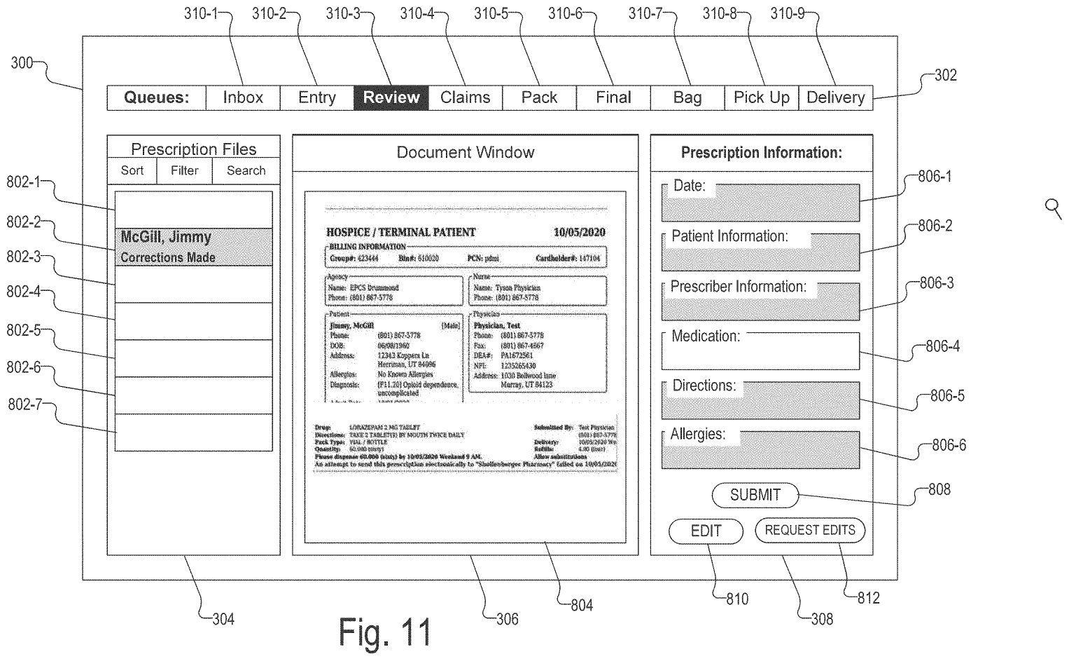

When the unapproved prescription file is returned to the entry queue for edits, system 100 may provide, in GUI 300, a visual indication that edits are needed and may visually indicate which information modules 806 need correction. FIG. 11 shows an illustrative implementation of GUI 300 when the entry queue is active. As shown, each prescription file identifier 502 for which corrections are needed is flagged to indicate that are corrections are needed. For example, prescription file identifier 502-2 includes a message that states, “Corrections Needed.” However, any other flag or visual indication may be used (e.g., an exclamation point, a different background color, etc.). When the flagged prescription file identifier 502-2 is selected in navigation window 304, the information module 806 with the “Not Approved” status (medication module 806-4) is visually identified and distinguished from the approved information modules 806, as shown in FIG. 10.

As shown, medication information module 806-4 is presented with a red background color (indicated by cross-hatching in FIG. 10), as was also indicated while the prescription file was in the review queue (see FIG. 9). In some examples, the approval status of approved information modules 806 may also be indicated, such as with a green background. In this way, a user can easily see which information module 806 needs to be corrected and quickly make the necessary edits. Upon making the edits, the user may select the submit button 510 to move the prescription file back to the review queue. When the prescription file is moved back to the review queue again, system 100 may automatically set the approval status of any information module 806 that was edited in the entry queue to “Needs Review.”

When the prescription file is returned to the review queue for review again, system 100 may provide a visual indication that edits have been made and may visually indicate which information modules 806 have been edited, as shown in FIG. 11. FIG. 11 shows an illustrative implementation of GUI 300 when the review queue is active. As shown, each prescription file identifier 802 for which corrections have been made is flagged to indicate that corrections were made. For example, prescription file identifier 802-2 includes a message that states, “Corrections Made.” However, any other flag or visual indication may be used (e.g., a check mark, a different text and/or background color, etc.). When a flagged prescription file identifier 802 is selected in navigation window 304, the corrected information module 806 is presented with a “Needs Review” status indicated, as explained above, and thus is visually identified and distinguished from the approved information modules 806.

As shown in FIG. 11, medication module 806-4 is presented with a white background color. In this way, a user can easily see which information module 806 has been corrected and has a “Needs Review” status. Thus, the user need only review that information module 806. Upon approving the edited information module 806 in the manner explained above, the user may select submit button 808 to move the prescription file to the next queue (e.g., the claims queue).

To enable this review and approval processing and visually indicate, in both the entry queue and review queue, which information modules 806 need review, which have been approved, and which need correction, system 100 may assign to each information module 806 a status value indicating the current approval status of the information module 806. In the entry queue and the review queue, the approval status of each information module 806 is visually indicated based on the assigned status value of the information module 806. When a prescription file is initially created, each information module 806 may be assigned a status value of “Needs Review” by default and thus each information module 806 is presented with a white background in the entry queue, as shown in FIG. 5. When information associated with an information module 806 is updated or changed system 100 may automatically change the status value of the information module 806 to “Needs Review.” In some examples, system 100 automatically changes the status value of an information module 806 only after completion of the entry queue (e.g., after submit button 510 has been selected). In this way, system 100 does not change the status value of an information module 806 in the entry queue when data is first entered into the data fields.

Referring again to FIG. 8, while the review queue is active, action window 308 may also include selectable options (not shown) to view the patient's profile, prescriber information, and/or information about the prescribed drug. The information may be presented, for example, in document window 306, action window 308, a pop-up window, or a new browser tab. Patient information may be pulled from a patient profile database within system 100. Prescriber information may be pulled from a prescriber profile database within system 100 and/or from prescriber system 208. Drug information may be pulled from a drug database, which may be maintained by system 100 or by a third-party service.

In some examples, system 100 requires review and approval of a clinical review module before advancing the prescription file to the next queue. The clinical review module may provide any clinical information or warnings that may be relevant to a pharmacist, such as duplicate therapies, contraindications, drug interactions, and/or whether any allowed refills are too soon or too many. In some examples, the clinical warnings may be generated based on drug information accessed from the drug database. In other examples, system 100 may be configured to identify duplicate therapies, such as when the same patient is prescribed the same medication (e.g., in the same prescription file or in multiple different prescription files). For example, system 100 may review all prescription files assigned to the same patient to identify duplicate therapies or drug interactions.

In some examples, system 100 may also include a fraud detection module. For example, system 100 may integrate with third party databases (e.g., a controlled substance database) to confirm that patient and/or prescriber assigned to the currently active prescription file is/are not suspected of fraudulent activity.

When a prescription file has been fully approved and the user has selected the submit button 808 to move the prescription file to the next queue (e.g., the claims queue), system 100 may submit, to the patient's insurance provider, an insurance claim for the prescribed medication identified in the prescription file. This may be performed in any suitable way. In some examples, system 100 is communicatively connected, by way of network 206, with a third party “switch” (not shown in FIG. 2) (e.g., RelayHealth Pharmacy Solutions or eRx Network) that routes the insurance claim (e.g., the prescription file and any associated documents and patient profile information, etc.) to the patient's insurance provider. The insurance provider may then accept or reject the claim and send notice of the approval or rejection back to system 100 by way of the switch.

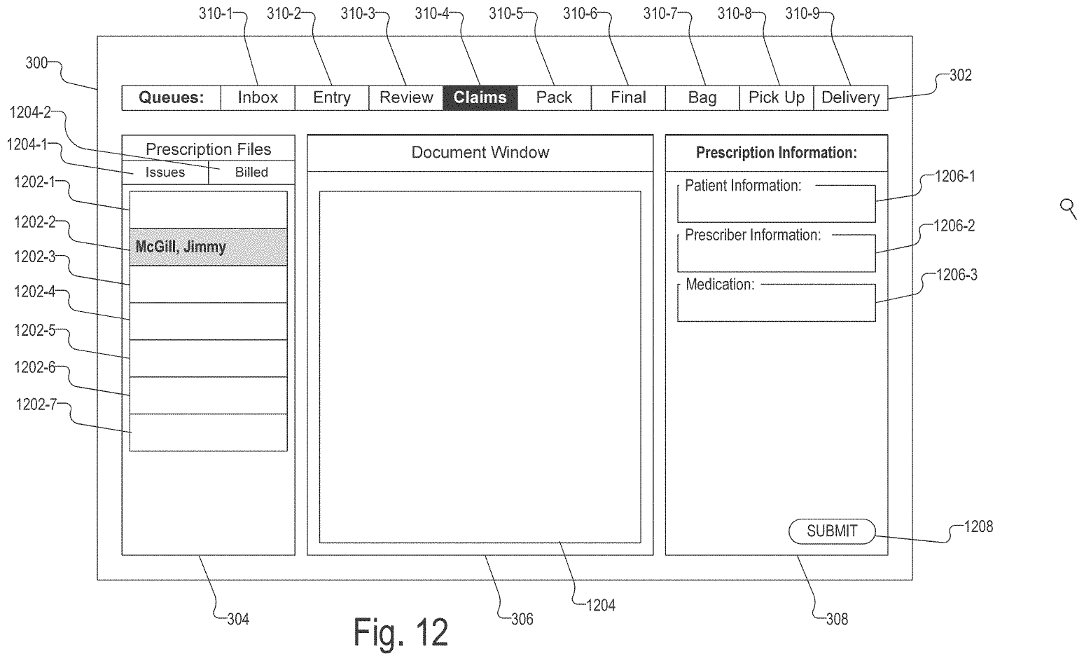

While the claims queue is active, system 100 may indicate, by way of GUI 300, the claim status (e.g., pending, accepted, rejected, etc.) of submitted insurance claims for prescription files. FIG. 12 shows an illustrative implementation of GUI 300 while the claims queue is active. As shown, navigation window 304 displays prescription file identifiers 1202 for prescription files for which insurance claims have been submitted. In some examples, navigation window 304 sorts prescription file identifiers based on the claim status of the prescription files. For example, navigation window 304 includes a first selectable option 1204-1 that may be selected to show prescription file identifiers 1202 for which the insurance claim has issues (e.g., has been rejected). Navigation window 304 also includes a second selectable option 1204-2 that may be selected to show prescription file identifiers 1202 for which the insurance claim has been accepted (e.g., billed). In some examples, navigation window 304 may also include a third selectable option (not shown) that may be selected to show prescription file identifiers 1202 for which the insurance claim is still pending (e.g., has not yet been reviewed, rejected, or approved by the insurance provider). In other examples, navigation window 304 may show prescription file identifiers 1202 for only those prescription files for which the insurance claims have been rejected (and, optionally, pending), and system 100 may automatically move accepted (e.g., billed) insurance claims to the next queue.

When a particular prescription file identifier 1202 is selected in navigation window 304 (e.g., prescription file identifier 1202-2), system 100 may show information about the insurance claim in document window 306 (e.g., insurance codes, any issues, reasons for rejections, notes, insurance provider documents, letters of rejection, etc.).

Action window 308 may provide options for a user to resolve the issues. For example, an insurance claim may be rejected by the insurance provider if the claim does not provide certain patient information (e.g., patient height, weight, drug allergies, diagnosis, etc.), prescription information (e.g., particular brand of medication is not covered), or prescriber information (e.g., name, contact info, Drug Enforcement Administration (DEA) number, etc.). Accordingly, action window 308 may include various fields and/or information modules 1206 (e.g., information modules 1206-1 to 1206-3) by which the user may enter the missing information for the insurance claim. In some examples, action window 308 may include a set of selectable options (e.g., tabs) to show, in action window 308, only a certain set of information modules. For example, a first selectable option may be selected to access and/or edit patient information, a second selectable option may be selected to access and/or edit prescription information, a third selectable option may be selected to access and/or edit prescriber information, and a fourth selectable option may be selected to access and/or edit transaction information. Action window 308 may include any other number of selectable options to access input fields for additional or alternative information. Alternatively, all input fields may be presented in the same window and may be accessed by scrolling through action window 308. Action window 308 also includes a selectable submit button 1208 to re-submit the insurance claim to the insurance provider after corrections have been made.

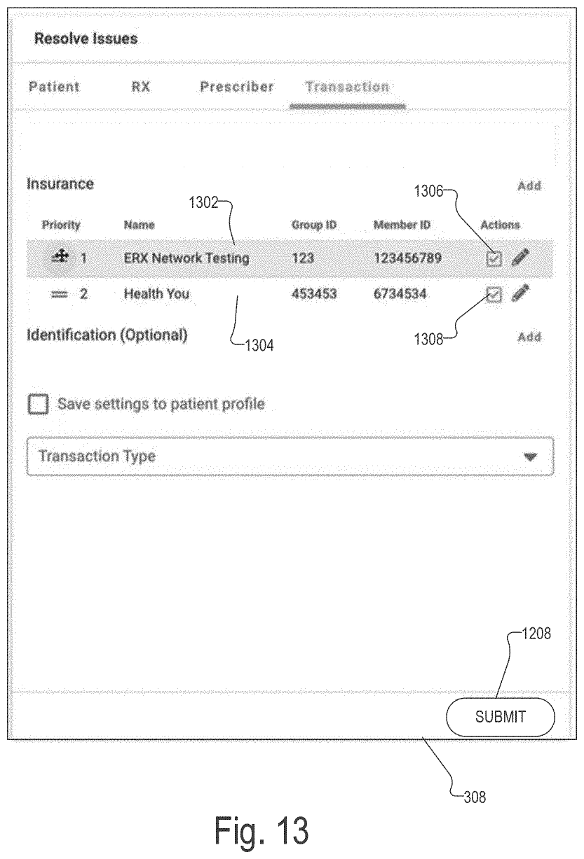

In some examples, the patient may be covered by multiple insurance policies. Accordingly, a transaction module of the action window 308 may allow the user to adjust the order in which the insurance policies will be billed, as shown in FIG. 13. FIG. 13 shows an illustrative implementation of action window 308 while the claims queue is active. As shown, each insurance policy is indicated on a separate line (e.g., line 1302 or line 1304). The user may adjust the order of billing insurance policies by rearranging/re-ordering the lines (such as with a drag-and-drop operation, etc.). Thus, the insurance policy on first line 1302 will be billed as the primary insurance policy and the insurance policy on second line 1304 will be billed as the secondary insurance policy. Additionally or alternatively, the user can select a selectable option on each line (e.g., a checkbox 1306 on first line 1302 ora checkbox 1308 on second line 1304) to indicate which insurance policy to bill and which to not bill (unchecked policies will not be billed).

Referring again to FIG. 12, in some examples, the information in the insurance claim that is missing or incorrect may be indicated in GUI 300 in a manner similar to indicating the approval status in the review queue and entry queue, as previously described. For example, based on information received from the insurance provider, system 100 may visually indicate, in GUI 300 while the claim queue is active, an information module 1206 in action window 308 for which edits or additional information are needed.

In the examples described above, the insurance claim is initially submitted to the insurance provider when the user selects submit button 808 when the review queue is active. In alternative examples, the insurance claim is first submitted to the insurance provider when the user selects submit button 1208 when the claims queue is active. Thus, the user may review and edit or correct information before the claim is submitted, thus preventing insurance claim issues and thereby speeding up the claim review process by the insurance provider.

In some examples, a user may select an option (not shown) in document window 306 or action window 308 to view or access information stored by system 100 and/or any other connected system (e.g., prescriber system 208) that may be helpful with resolving any insurance claims issues, such as patient information and/or prescriber information. Thus, a user can quickly and easily correct any insurance claim and billing issue since all of the information needed to address the insurance claim is readily available and can be input through system 100 without the user navigating away from his or her place in working through the queues.

If the insurance claim is rejected again, the prescription file identifier 1202 will be presented in navigation window 304 of the claims queue. When the insurance claim is accepted and the insurance provider has been billed, system 100 moves the prescription file to the next queue (e.g., the pack queue).

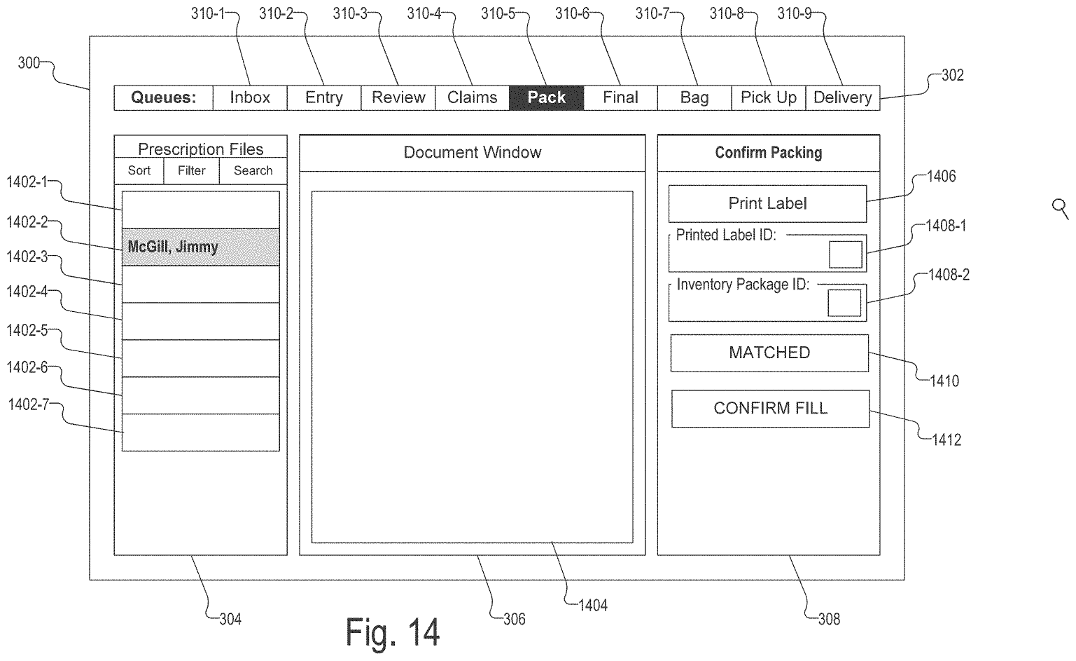

When the prescription file is assigned to the pack queue, labels may be printed, the drugs may be removed from the shelf, and the drugs may be placed (“packed”) in a container (e.g., medicine bottles, vials, pill bottles, etc.), and the packed container may be labeled with the printed label. FIG. 14 shows an illustrative implementation of GUI 300 while the pack queue is active. As shown, navigation window 304 includes a plurality of prescription file identifiers 1402 (e.g., prescription file identifiers 1402-1 to 1402-7) each representing a unique prescription file for which the associated drug is ready for packing. In some examples (not shown), prescription file identifiers 1402 may be grouped by patient, so that navigation window 304 shows one or more patient identifiers instead of prescription file identifiers. When a user selects a patient identifier in navigation window 304, the user may select one or more prescription file identifiers 1402 grouped with that patient identifier for which the prescribed medicine will be packed. Document window 306 may present any document 1404 or information associated with each selected prescription file identifier, such as the original prescription, the packing label to be printed, and/or pictures of the drugs to pack.

Action window 308 (or, alternatively, document window 306) includes a selectable option 1406 to print the label for each selected prescription file identifier 1402. Once the label has been printed, the user may manually input the printed label ID into a label module 1408-1 or select a selectable option to scan the printed label (e.g., a bar code or QR code on the label) with a scanning device (e.g., a barcode scanner, a camera, etc.) coupled to or included in the user device to capture the prescription file number. The user may also manually input the inventory package ID into a package module 1408-2 or select a selectable option to scan the inventory package (e.g., scan a bar code or QR code) in inventory from which the drug will be taken to fill the patient's container. System 100 may then compare the medicine information associated with the scanned label with the medicine information associated with the scanned inventory package to confirm whether the medicine pulled from inventory and packed in the container is the correct medicine for the particular prescription. System 100 may then indicate (e.g., in match module 1410) whether the scanned information is a match (e.g., “Matched” or “No Match”). If the information is a match, system 100 may activate a selectable confirm button 1412 that the user may select to proceed to the next queue (e.g., the final queue).

In some examples, the user may also perform a short fill (e.g., dispense less than the prescribed quantity of the drug, such as when inventory is low). Accordingly, the user may input, by way of document window 306 or action window 308, the actual quantity of drug that was dispensed.

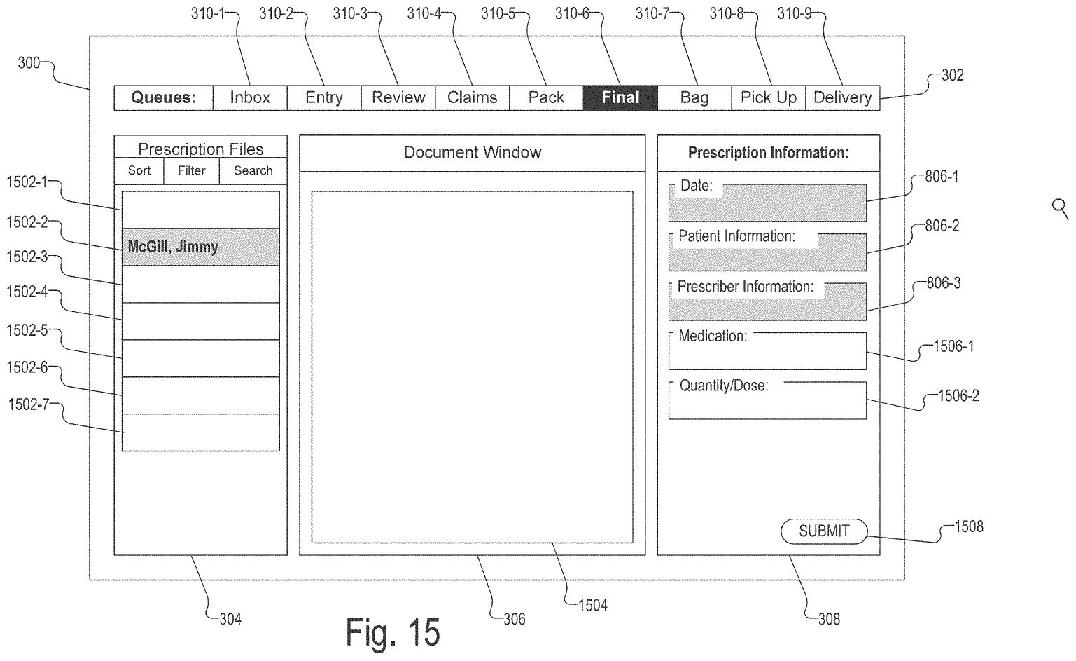

The final queue is another review queue (similar to the review queue) that allows a user to confirm that the correct drug in the correct amount has been placed in the correct container. FIG. 15 shows an illustrative implementation of GUI 300 when the final queue is active. As shown, navigation window 304 includes a plurality of prescription file identifiers 1502 (e.g., prescription file identifiers 1502-1 to 1502-7) each representing a unique prescription file that is ready for final review and approval for dispensing of the packed medication to the patient. Document window 306 may display a document 1504 or any other information associated with the selected prescription file identifier 1502.

Action window 308 allows the user to review and confirm any additional information that has been added to the selected prescription file since the review queue was completed. For example, action window 308 may include information modules 806 previously presented when the entry queue and review queue were active. Action window 308 may also include additional information modules 1506 that provide information about the drug packed in the container. FIG. 15 shows that information modules 1506 include a medication module 1506-1 and a quantity/dosage module 1506-2 in addition to information modules 806-1 to 806-3. Action window 308 may include any additional or alternative information modules 1506 as may suit a particular implementation. Information modules 806 indicate their approved status, which was previously set when the review queue was completed.

In action window 308, the user may interact with (e.g., touch) module information modules 1506 to approve the information in each information module 1506 in any manner as described above for information modules 806. For example, the user may approve medication module 1506-1 and quantity/dose module 1506-2 to confirm that the correct drug, in the correct quantity, is in the container. If the information is not correct, the user may provide input to mark the approval status of information modules 1506 as “Not Approved” to indicate that corrections are needed.

If information modules 1506 are marked with a “Not Approved” status, system 100 assigns the prescription file to the pack queue again when the user selects a submit button 1508. While the pack queue is active, system 100 may flag each prescription file identifier 1802 for a prescription file that needs correction (e.g., has a “Not Approved” status), and may indicate in action window 308 of the pack queue the particular information that is incorrect (e.g., the quantity of pills is wrong). System 100 may flag the particular prescription file identifier 1802 and information for correction in any manner described herein. The pack queue process may then be repeated, and upon completion of the pack queue, system 100 assigns the prescription file to the final queue again.

As mentioned above, while the final queue is active, system 100 maintains the previously set approval state of information modules 806 so that the user need not confirm information that was previously confirmed in the review queue. In other examples, system 100 resets, when the prescription file is moved to the pack queue or the final queue, some or all of the information previously confirmed in the review queue so that the user must reconfirm the information that was previously confirmed. In some examples, system 100 requires reconfirmation only for select information modules 806 (e.g., quantity/dose module 1506-4) and/or for select patients (e.g., patients with multiple prescriptions being filled, patients with certain medical conditions, or patients prescribed certain drugs).

In further examples, the review queue may be combined into the final queue so that only one review is performed. In these examples, the review queue is omitted so that system 100 advances prescription files from the entry queue directly to the claims queue. If the user indicates that corrections or edits are needed, system 100 returns the prescription file to the appropriate queue (e.g., the entry queue for corrections of information modules 806 and the pack queue for corrections of information modules 1506). In some examples, a user may toggle the review queue on and off as desired.

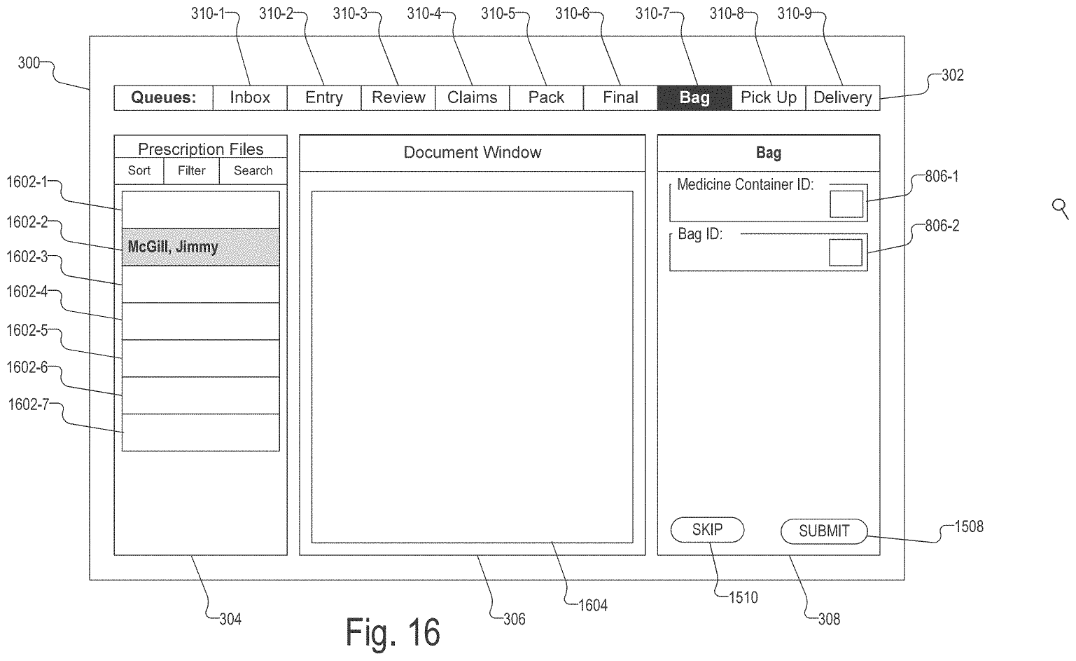

If all information modules 1506 have an “Approved” status when submit button 1508 is selected, system 100 moves the prescription file to the bag queue. The bag queue is used to indicate a storage bag in which the packed container will be stored if the patient will not pick up the filled prescription immediately. FIG. 16 shows an illustrative implementation of GUI 300 when the bag queue is active. As shown, navigation window 304 includes a plurality of prescription file identifiers 1602 (e.g., prescription file identifiers 1602-1 to 1602-7) each representing a unique prescription file that has passed the final review for which the filled container is ready to be bagged for later pick-up. Document window 306 may display a document 1604 or any other information associated with the selected prescription file identifier 1502 (e.g., a photo of the bag, a map of the bag location, a photo of the packed container, etc.). In some examples, document window 306 is omitted while the bag queue is active.

In action window 308, the user may manually input the printed label ID into a label module 1608-1 or select a selectable option to scan the printed label on the packed medicine container (e.g., a bar code or QR code on the label) with a scanning device (e.g., a barcode scanner, a camera, etc.) coupled to or included in the user device to capture the prescription file number. Alternatively, system 100 may use the printed label ID that was input or scanned in the pack queue. The user may also manually input a bag ID into a bag module 1608-2 or select a selectable option to scan the bag ID (e.g., scan a bar code or QR code) in inventory from which the drug will be taken to fill the patient's container. The user may also select which packed medicine container(s) are placed in each scanned bag. A user may later look up the patient's bag ID in the pick up queue or the delivery queue, as will be explained below. When a prescription file is assigned to a bag and a submit button 1608 is selected, system 100 moves the prescription file to the pick up queue or the delivery queue. In some examples, as shown in FIG. 16, action window 308 includes a skip button 1610 that may be selected to skip bagging and proceed to directly to the pick up queue or delivery queue. The bagging step may be skipped, for example, when the patient is at the pharmacy and ready for immediate pick up of the medicine.

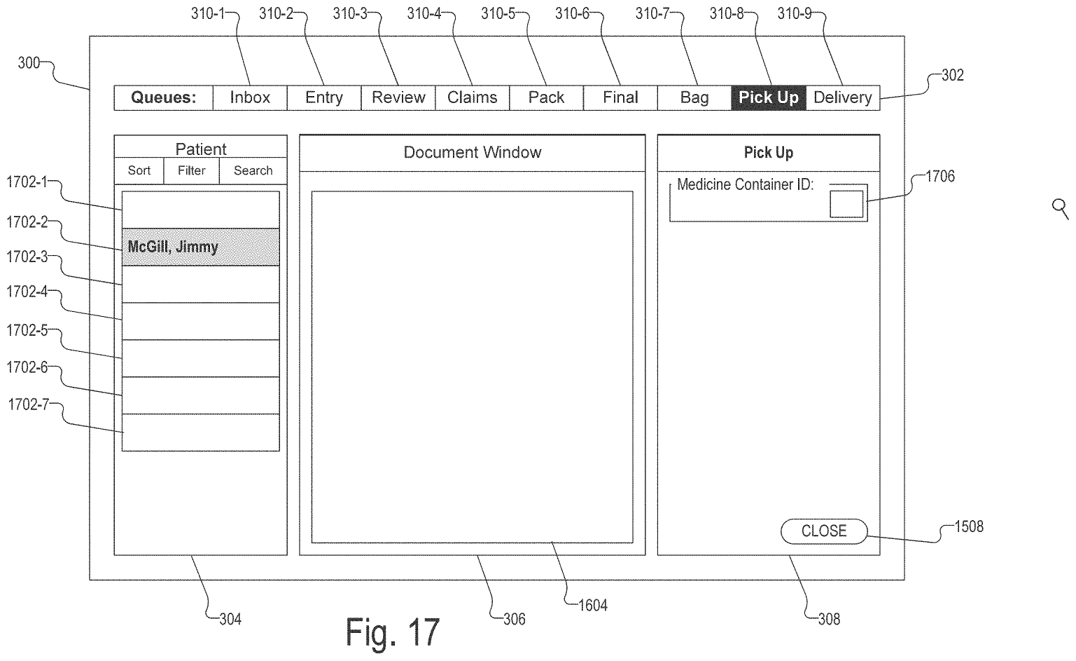

FIG. 17 shows an illustrative implementation of GUI 300 when the pick up queue is active. As shown, navigation window 304 includes a plurality of prescription file identifiers 1702 (e.g., prescription file identifiers 1702-1 to 1702-7) each representing a unique prescription file for which the filled container is ready for pick-up by the patient. Navigation window 304 includes sort, filter, and search options by which a user may search for a particular prescription file identifier 1702 based on the patient's name, date-of-birth, and/or other information. Upon a user selecting a particular prescription file identifier 1702, document window 306 system 100 may display a document 1704 or other information associated with the selected prescription file identifier 1702, such as the status of the order and the location (e.g., bag ID and location) so the user can easily retrieve the medicine container for the patient. In some examples, action window 308 of the pick up queue includes a selectable option 1706 to scan a barcode or label of the bag or the packed container at pick up. In response to scanning the barcode or label of the bag or packed container, system 100 may automatically mark the medicine as delivered and the close the prescription file out of the queues. Alternatively, the user may mark the medicine as delivered to the patient, such as by selecting a close button 1708. In some examples, system 100 may communicate with the pharmacy checkout and payment system to update a status of prescription files at checkout. For example, When the customer pays for the medication through the checkout system and picks up the medication, system 100 marks the medicine as delivered and closes the prescription file out of the queues.

In some examples, the pharmacy or a third party (e.g., a delivery service) may deliver the medication to the patient rather than wait for the patient to pick up the medicine at the pharmacy location. To this end, GUI 300 may allow a user to mark a prescription file to be delivered. For example, when the entry queue (or any other queue) is active, action window 308 may include a selectable delivery option for a user to select to indicate that the medicine is to be delivered to the patient. Action window 308 may also include options to input delivery information and instructions, such as delivery times, delivery address, patient or caregiver name, etc. When a prescription file has been marked for delivery, system 100 moves the prescription file from the final queue or the bag queue to the delivery queue (rather than to the pick up queue).

In the delivery queue, system 100 may display delivery information for medicines to be delivered, such as a list of deliveries, addresses, delivery times, notes for deliveries, routes, current location of the delivery truck, estimated delivery times, status of deliveries, and the bag ID. In some examples in which the pharmacy is a closed-door or mail order pharmacy, the delivery queue may indicate when medication has been shipped, may include tracking information, and may indicate when the medication has been successfully delivered.

In some examples, a mobile computing device (e.g., a mobile phone, a tablet computer, a laptop computer, etc.) may execute an application to access the delivery queue information. The application may be a browser-based application, or it may be a standalone application installed on the mobile computing device. Thus, a delivery driver (e.g., user 216-3) may use the mobile computing device (e.g., user device 204-3) in the delivery of medications to patients, such as to view routes, schedules, and to confirm successful delivery of medications. For example, the delivery user may scan (e.g., with a dedicated scanner connected to the mobile computing device or a camera in the mobile computing device) the labels on the medicine containers when the medicine is loaded onto the delivery truck and/or when the medicine is delivered to the patient.

In some examples, system 100 may communicate with a global positioning system (GPS) unit on the mobile device to update a status of prescription files. For example, when a GPS location of the mobile device matches a location of the delivery address, system 100 marks the medicine as delivered and closes the prescription file out of the queues.

In some examples, a prescriber computing device (e.g., prescriber system 208) may also execute an application to interact with one or more features provided by system 100. The prescriber application may be a browser-based application, or it may be a standalone application installed on the prescriber computing device. For example, prescriber system 208 may be used by the prescriber (or prescriber's office staff) to electronically upload patient and prescription documents, input patient and prescription information (e.g., in the entry queue), receive notification that the prescription has been approved by the pharmacist (e.g., upon the review queue and/or final queue), to address clinical review issues identified during the review queue, to receive notification of and/or address insurance billing issues, to receive notification that the prescription has been picked up or delivered, and/or to securely communicate with the pharmacy.

In some examples, a patient computing device (e.g., a mobile phone, a tablet computer, a personal computer, etc.) may execute an application to interact with one or more features provided by system 100. The patient application may be a browser-based application, or it may be a standalone application installed on the mobile computing device. For example, patient device 210 may be used by patient 212 to request a new fill or refill a prescription, electronically upload patient and prescription documents (e.g., scan paper prescriptions, health charts, etc.), receive notification of and/or address insurance billing issues, review pricing information (e.g., estimated costs for the medicine, costs of alternative or generic medications), pay for medication, receive notification that the prescription is available for pick up, track delivery of the prescription, securely communicate with the pharmacist, review directions for taking the medication, view drug information (e.g., side effects, etc.), view photos of the drugs, and the like.

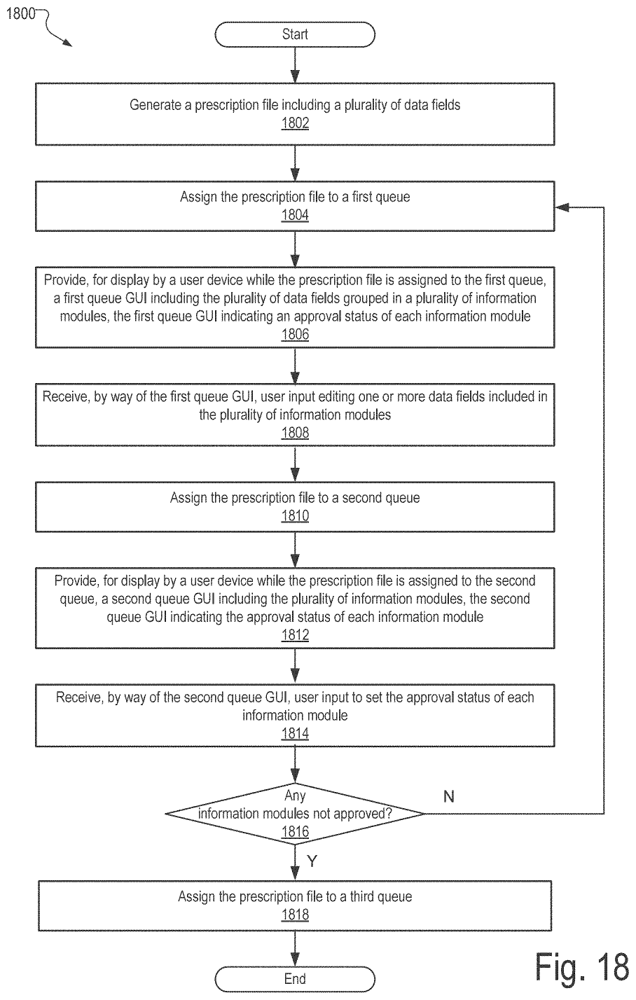

FIG. 18 shows an illustrative method 1800 of using GUI 300 to review and confirm information in a prescription file. While FIG. 18 shows illustrative operations according to one embodiment, other embodiments may omit, add to, reorder, and/or modify any of the operations shown in FIG. 18. One or more of the operations shown in FIG. 18 may be performed by system 100, any components included therein, and/or any implementations thereof. Any of the operations shown in FIG. 18 may be performed in any way described herein.

At operation 1802, system 100 generates a prescription file including a plurality of data fields.

At operation 1804, system 100 assigns the prescription file to a first queue (e.g., the entry queue or the claims queue).

At operation 1806, system 100 provides, for display by a user device while the prescription file is assigned to the first queue, a first queue GUI (e.g., GUI 300 when the entry queue or the pack queue is active). The first queue GUI includes the plurality of data fields grouped in a plurality of information modules (e.g., information modules 506 or 1206). The first queue GUI indicates an approval status of each information module (e.g., “Needs Review,” “Approved,” or “Not Approved”).

At operation 1808, system 100 receives, by way of the first queue GUI, user input editing one or more data fields included in the plurality of information modules.

At operation 1810, system 100 assigns the prescription file to a second queue (e.g., the review queue or the final queue).

At operation 1812, system 100 provides, for display by a user device while the prescription file is assigned to the second queue, a second queue GUI (e.g., GUI 300 when the review queue or the final queue is active). The second queue GUI includes the plurality of information modules and indicates the approval status of each information module.

At operation 1814, system 100 receives, by way of the second queue GUI, user input setting the approval status of each information module.

At operation 1816, system 100 determines whether any information modules have a “Not Approved” status (or if all information modules have an “Approved” status). If any information modules have a “Not Approved” status, system 100 returns to operation 1804, and processing through the first queue is repeated. If no information modules have a “Not Approved” status, or if all information modules have an “Approved” status, system 100 proceeds to operation 1818.

At operation 1818, system 100 assigns the prescription file to a third queue (e.g., the claims queue, the bag queue, the pick up queue, or the delivery queue).

System 100 allows multiple different users at multiple different locations to process a prescription file. For example, a pharmacy technician, intern, or other data entry clerk may access the inbox queue and the entry queue from any remote location and enter information to create a prescription file. A pharmacist or other user may then review the prescription file information in the review queue from any physical location. Insurance information and insurance claim issues may also be reviewed and resolved by any suitable user from any remote location. Thus, many tasks that have traditionally been performed on-site at a pharmacy location may be performed remotely and with personnel other than on-site personnel, thereby freeing up computing resources at the pharmacy location. In some examples, system 100 may include a feature to “request help” to process a large batch of prescriptions. In response to such a call, one or more remote workers may receive a notification requesting help and may then log-in to system 100 from their remote location and begin processing prescription documents and prescription files. In this way, an on-site pharmacist may handle and manage other tasks associated with filling and dispensing medications while other remote workers handle the data entry tasks.

In some examples, system 100 may provide location, user, and/or role-based rights permissions for any one or more features (e.g., queues or tasks within queues) provided by system 100. For example, the review queue and pack queue may be restricted to users that are designated as a pharmacist, the entry queue may be unrestricted, and the pack queue, final queue, bag queue, and/or pick up queue may be accessible only by user devices that are physically located at the pharmacy location and/or by certain users. The rights permissions may be in any suitable form, such as edit access, read-only access, or no access (e.g., do not appear on the GUI).

In some examples, system 100 may also enable certain information (e.g., information modules) to be “locked” so that the information cannot be edited. For example, a prescription file may be automatically locked while it is actively selected in a navigation window 304 on a user device 204 so that only the user device 204 on which the prescription file is actively selected may make edits to the prescription file. Other user devices may be locked from editing and/or viewing the prescription file until the lock is removed, such as when the prescription file is closed out and no longer active on the user device 204. This may prevent multiple different users from editing the same prescription file at the same time and inadvertently overriding information or edits made by each other.

System 100 provides a fully end-to-end paperless prescription processing ecosystem, from the initial prescription generation to pick up/delivery of the medicine. Additionally, system 100 may automatically provide a notification to the prescriber, the pharmacy, and/or the patient when the medicine has been picked up or delivered. Furthermore, system 100, rather than user devices 204, connects with the various different third parties that are sometimes necessary to process a prescription. This reduces bandwidth for each user device 204 of the pharmacy and significantly speeds up the process for the pharmacy by eliminating the number of connection handshakes needed and reducing the need to log in to multiple different systems. For instance, the pharmacy user device 204 need not communicate directly with multiple different third parties, such as the prescriber, third party switches or insurance entities, controlled substance databases, patient devices, or other associated pharmacies. Rather, all information needed by a pharmacy user device 204 may be channeled through system 100, which maintains and manages the connections with the third-party systems.

Moreover, the configuration of system 100 allows one or more features of system 100 to be accessible and used remotely and without a remote desktop service or virtual private network (VPN). Thus, even mobile devices (e.g., mobile phones, smartphones, tablet computers, etc.) can access features of system 100. Furthermore, because system 100 receives and stores prescription documents electronically, certain tasks (e.g., the entry queue tasks) can be performed anywhere, even remotely from the pharmacy and with the use of mobile devices. Thus, system 100 may reduce the number of staff that need to be physically present at the pharmacy location. System 100 also provides a simple yet elegant method for reviewing and approving prescription files and indicating when and where corrections are needed.

In some examples, system 100 and/or one or more other prescription management systems may be included in a connected prescription platform that provides real-time visibility to the status of prescription medication provisioning. An illustrative connected prescription platform for medication provisioning will now be described.

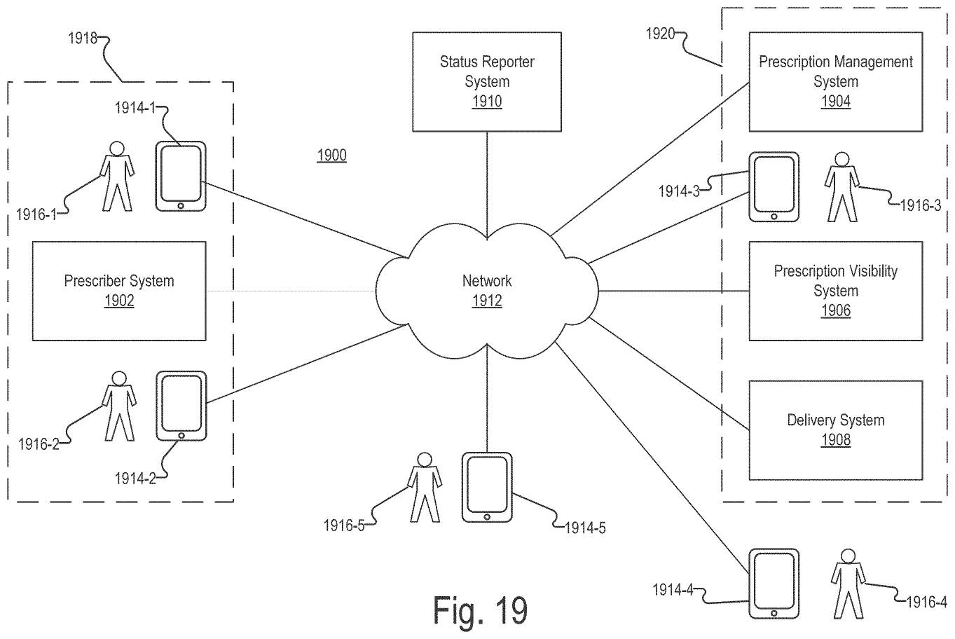

FIG. 19 shows an illustrative architecture of a connected prescription platform 1900 (“platform 1900”). The architecture shown is merely illustrative, as other architectures that achieve the same or similar visibility features may be used. As shown, platform 1900 includes a prescriber system 1902, a prescription management system 1904, a prescription visibility system 1906 (“visibility system 1906”), a delivery system 1908, and a status reporter system 1910 (“status system 1910”) communicatively connected to one another by way of a network 1912. Network 1912 may be a local area network, a wireless network (e.g., Wi-Fi), a wide area network, the Internet, a cellular data network, and/or any other suitable network. Data may flow between components connected to network 1912 using any communication technologies, devices, media, and protocols as may serve a particular implementation. In some examples, network 1912 is implemented by multiple different networks. In some examples, network 1912 is implemented by network 206 of FIG. 2.

A plurality of user devices 1914 (e.g., user devices 1914-1 to 1914-5) are associated with one or more of systems 1902-2210 and communicatively connected to systems 1902-2210 by way of network 1912 or a different network. User devices 1914 are configured to provide users 1916 (e.g., users 1916-1 to 1916-5) with access to any one or more of systems 1902-2210 (e.g., to data maintained by systems 1902-2210). User devices 1914 may be implemented by any user device described herein (e.g., a user device 204).

Users 1916-1 to 1916-4 may together form a care team for providing medical care to patient 1916-5. For example, user 1916-1 may be a nurse, user 1916-2 may be a physician, user 1916-3 may be a pharmacist, and user 1916-4 may be a delivery driver. While FIG. 19 shows that platform 1900 includes five user devices 1914 and five users 1916, platform 1900 may include any other number of user devices 1914 and users 1916 as may serve a particular implementation.

User devices 1914-1 and 1914-2 may be associated with a medical facility 1918, and user devices 1914-3 and 1914-4 may be associated with a pharmacy 1920. For example, user devices 1914-1 and 1914-2 may be physically located at medical facility 1918, used by user 1916-1 (e.g., a nurse) and user 1916-2 (e.g., a physician) who are associated with medical facility 1918 (e.g., employed by or located at medical facility 1918), and/or maintain and/or provide access to data (e.g., patient data, medication data, prescription data, etc.) associated with medical facility 1918. User devices 1914-3 and 1914-4 may be physically located at pharmacy 1920, used by pharmacist 1916-3 or delivery driver 1916-4, and/or maintain and/or provide access to data (e.g., patient or customer data, inventory data, etc.) associated with pharmacy 1920.

User devices 1914 may store and execute an application that provides a user 1916 with access to information and data maintained by the particular system with which it is associated (e.g., prescriber system 1902, prescription management system 1904, visibility system 1906, delivery system 1908, and/or status system 1910). The application may be any type of application as may suit a particular implementation. For example, the application may be a client-side application (e.g., a “thin client application” such as a mobile application) locally installed on user devices 1914. As another example, the application may be a web application accessible through a browser application installed on user devices 1914.

In some examples, patient 1916-5 may use user device 1914-5 to communicate with pharmacy 1920, such as to ask pharmacist 1916-3 questions, request medication refills, provide insurance information, provide delivery information to delivery driver 1916-4, and/or to view the delivery status (e.g., estimated delivery time) of medication. Patient 1916-5 may additionally or alternatively use user device 1914-5 to communicate with medical facility 1918, such as to request new medication, update physician 1916-1 with the effectiveness of medication or adjust dosage, request assistance from physician 1916-1 or nurse 1916-2, and/or for any other suitable purpose. In some examples, user 1916-5 may also use user device 1914-5 to view the current status of a medication order or prescription. Patient 1916-5 may additionally or alternatively use user device 1914-5 to communicate with delivery driver 1916-4, such as to provide delivery instructions and/or coordinate timing of the delivery.

Prescriber system 1902 may be associated with (e.g., used by, accessible by, etc.) medical facility 1918, such as a hospital, a surgical facility, a medical clinic, a doctor's office, a dental office, a nursing home, a hospice facility, a rehab facility, an assisted living facility, or any other similar medical or healthcare facility. Prescriber system 1902 may be used by a medical provider to maintain and manage patient data and records, order and schedule delivery of medication, generate and manage prescriptions, submit prescriptions to pharmacy 1920, and/or perform any other suitable operations. For example, user 1916-1 (e.g., a nurse) may use user device 1914-1 to interact with prescriber system 1902 to order medication for patient 116-5, and user 1916-2 (e.g., a physician or nurse practitioner) may use user device 1914-2 to interact with prescriber system 1902 to review and approve the medication order and submit a prescription to prescription management system 1904. Prescriber system 1902 may be implemented by hardware and/or software components (e.g., processors, memories, communication interfaces, instructions stored in memory for execution by the processors, etc.), and may be physically located locally on-site at medical facility 1918, distributed across multiple locations associated with medical facility 1918, and/or based in the cloud. In some examples, prescriber system 1902 is implemented by prescriber system 208.

Prescription management system 1904 may be associated with pharmacy 1920 and may facilitate management, by way of an application executed by user device 1914-3 and/or one or more other user devices not shown in FIG. 19, of prescription fulfillment, from receipt of the prescription from prescriber system 1902 to filling the prescription and dispensing the medication or sending the medication out for delivery (e.g., by delivery driver 1916-4) to patient 1916-5. Upon receiving a prescription from prescriber system 1902, prescription management system 1904 may be used to generate a prescription file based on the information contained in the received prescription. Prescription management system 1904 may be implemented by one or more computing devices (e.g., servers, personal computers, user devices, etc.) and may be physically located locally on site at pharmacy 1920, distributed across multiple locations associated with pharmacy 1920, and/or based in the cloud. In some examples, prescription management system 1904 is implemented by prescription management system 100 described herein. In alternative examples (not shown), prescription management system 1904 is not communicatively connected to status system 1910 and/or to prescriber system 1902. In such examples, prescription management system 1904 may be a legacy prescription management system or may be provided by a third party in such a way that it is not communicatively integrated with status system 1910 and/or prescriber system 1902.

Visibility system 1906 may be associated with pharmacy 1920 and may provide to a user (e.g., user 1916-3) the status of medications ordered by medical facility 1918 and the status of prescriptions that have been received by pharmacy 1920. As will be explained below in more detail, visibility system 1906 may communicate with status system 1910 to receive the current status of orders requested by prescriber system 1902, the status of prescriptions received and processed by prescription management system 1904, and the status of medications delivered or scheduled for delivery. Visibility system 1906 may be implemented by one or more computing devices (e.g., servers, personal computers, user devices, etc.) and may be physically located locally on site at pharmacy 1920, distributed across multiple locations associated with pharmacy 1920, and/or based in the cloud.

Delivery system 1908 may be associated with pharmacy 1920 and may be used by user 1916-4 (e.g., a delivery driver) to view delivery routes and schedules to physically deliver medications to patients (e.g., to patient 1916-5). User 1916-4 may interact with delivery system 1908 by way of user device 1914-4 to indicate the successful delivery of medications. In some examples, delivery system 1908 may use a tracking service (e.g., GPS) on user device 1914-4 to determine a current location of user 1916-4, to estimate, based on historical delivery data, traffic patterns, distance to scheduled stops, etc., a delivery time for the medication, and/or to confirm successful delivery of the medication to patient 1916-5. Delivery system 1908 may be implemented by one or more computing devices (e.g., servers, personal computers, user devices, etc.) and may be physically located locally on site at pharmacy 1920, distributed across multiple locations associated with pharmacy 1920, and/or based in the cloud.

While the example of FIG. 19 shows that platform 1900 has one each of systems 1902-2210, platform 1900 may include any number of systems 1902-2210 associated with any number of medical facilities and pharmacies as may serve a particular implementation. Moreover, while the example of FIG. 19 shows that prescription management system 1904, visibility system 1906, and delivery system 1908 are separate systems, any two or more of these systems may be combined into a single system. For example, visibility system 1906 and delivery system 1908 may be implemented as a single system with both visibility features and delivery features.

Status system 1910 is configured to track the real-time status of medication provisioning so that a user 1916 within the care team and/or patient 1916-5 may view, by way of a user device 1914, the real-time status of the medication provisioning. Status system 1910 may track the status of medication provisioning end-to-end from the initial order at medical facility 1918, through processing and dispensing at pharmacy 1920, to delivery to patient 1916-5. For example, status system 1910 may track and log triggering events that occur with respect to a prescription order at prescriber system 1902, prescription management system 1904, prescription validity system 1906, and/or delivery system 1908. Upon the occurrence of triggering events, status system updates the status of the prescription order along with a timestamp of the event or status change. The status change may be pulled by or pushed to user devices 1914. In this way, black holes that delay the provisioning of medication can be easily eliminated by providing everyone in the care team with on-demand real-time status of the prescription order. This may also increase accountability for the care team by clearly indicating which care team member is responsible for moving the order to the next stage.

Status system 1910 may be implemented by one or more computing devices (e.g., servers, personal computers, user devices, etc.) and may be physically located locally on site at medical facility 1918 or pharmacy 1920, distributed across multiple locations, and/or based in the cloud (e.g., a remote server or computing system). In some examples, status system 1910 includes a database (e.g., an SQL database) and an API (e.g., a RESTful API using FHIR or JSON) by which prescriber system 1902, prescription management system 1904, visibility system 1906, and delivery system 1908 may connect to status system 1910.

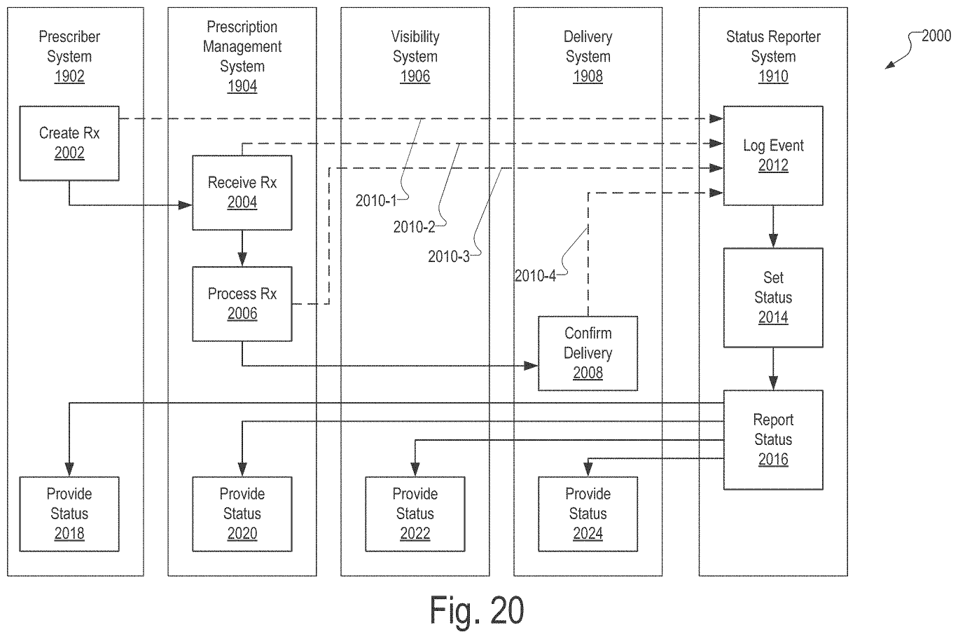

FIG. 20 shows an illustrative method 2000 of real-time reporting of the status of prescribed medication provisioning. While FIG. 20 shows illustrative operations according to one embodiment, other embodiments may omit, add to, reorder, and/or modify any of the operations shown in FIG. 20.

At operation 2002, prescriber system 1902 creates a prescription order using prescriber system 1902. Operation 2002 may be performed in any described herein. For example, as explained above, a user may interact with prescriber system 1902 to create a new prescription order, such as by entering patient information (e.g., name, address, birthdate, gender, medical condition, etc.), drug information (e.g., drug to be prescribed, dosage, instructions, etc.), and the patient's requested pharmacy (e.g., pharmacy 1920).

At operation 2004, prescription management system 1904 receives the new prescription and may respond to prescriber system 1902 with an acknowledgment that the new prescription was received. Operation 2004 may be performed in any suitable way.

At operation 2006, prescription management system 1904 processes the received prescription to prepare the medication for delivery to patient 1916-5. Operation 2006 may be performed in any suitable way, including any way described herein with respect to system 100. For example, prescription management system 1904 may generate a prescription file based on the received prescription, process the prescription file through various prescription file queues, and, upon confirmation of packing of the medication for delivery, transmit the prescription file to delivery system 1908.

At operation 2008, delivery system 1908 provides the prescription file for use by user 1916-4 to deliver the medication to patient 1916-5 and confirms successful delivery of the medication. Operation 2008 may be performed in any suitable way, including in any way described herein.

When each of operations 2002 to 2008 is performed, an event notification 2010 is transmitted to status system 1910. The event notification identifies at least the prescription order associated with the event. At operation 2012, status system 1910 logs the event for the particular prescription order, including the event type and a timestamp for the event, in response to receiving the event notification 2010. In some examples, the event notification 2010 includes information identifying the type of event. In other examples, status system infers the type of event based on the entity that transmitted the event notification 2010 and/or a current status of the prescription order.

To illustrate, in response to creating the prescription order (operation 2002), prescriber system 1902 transmits a new prescription order event notification 2010-1 to status system 1910. In response to acknowledging receipt of the new prescription order (operation 2004), prescription management system 1904 transmits a prescription order received event notification 2010-2 to status system 1910. In response to processing the new prescription (operation 2006), prescription management system 1904 transmits a prescription processed event notification 2010-3 to status system 1910. In response to confirmation of delivery of the prescribed medication (operation 2008), delivery system 1908 transmits a delivery confirmation event notification 2010-4 to status system 1910.

At operation 2014, status system 1910 sets, based on (e.g., in response to) logging the event, the status of the prescription order identified in the event notification. Operation 2014 may be performed in any suitable way. For example, upon logging prescription order received event notification 2010-2 from prescription management system 1904, status system 1910 may set the status of the prescription order as “pharmacy processing.”

At operation 2016, status system 1910 reports, in response to setting the status of the prescription order, the status of the prescription order to prescriber system 1902, prescription management system 1904, visibility system 1906, and delivery system 1908. Operation 2016 may be performed in any suitable way. In some examples, the status is reported in response to a status request from the recipient system. In other examples, status reporter system 1910 pushes the status to each of the recipient systems.

At operations 2018, 2020, 2022, and 2024, prescriber system 1902, prescription management system 1904, visibility system 1906, and/or delivery system 1908, respectively, provides an indication of the status of the prescription order. Operations 2018 to 2024 may be performed in any suitable way. In some examples, the status is provided by way of an application executed by a user device 1914 associated with the respective system. Illustrative examples of providing a status of a prescription order by way of an application executed by a user device 1914 will be described below in more detail. Additionally or alternatively, the status may be provided by sending a message to a user device 1914 (e.g., a text message, an email message, an automated voice message, etc.).

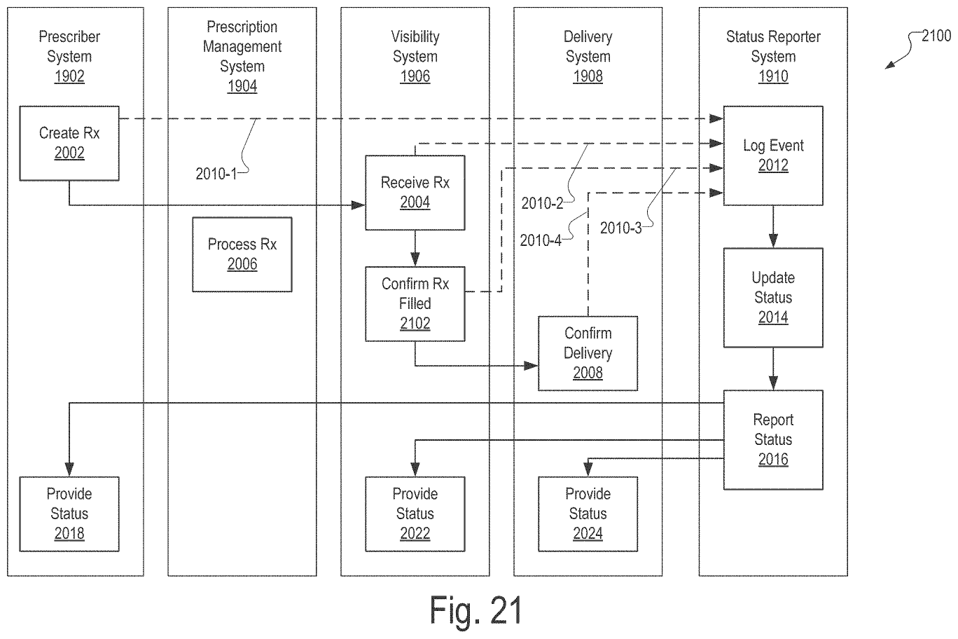

FIG. 21 shows another illustrative method 2100 of real-time reporting of the status of medication provisioning. While FIG. 21 shows illustrative operations according to one embodiment, other embodiments may omit, add to, reorder, and/or modify any of the operations shown in FIG. 21. Method 2100 is similar to method 2000 except that, in method 2100, prescription management system 1904 is not communicatively integrated with prescriber system 1902 and/or status system 1910. For instance, prescription management system 1904 may be a legacy prescription management system. Accordingly, new prescription orders created by prescriber system 1902 in operation 2002 are transmitted to visibility system 1906 (rather than to prescription management system 1904). Operation 2004 is then performed by visibility system 1906 to acknowledge receipt of the prescription order.

At operation 2006, prescription management system 1904 processes the received prescription to prepare the medication for delivery to patient 1916-5.

At operation 2102, visibility system 1906 confirms that the prescription has been processed and is ready for delivery to patient 1916-5. Operation 2102 may be performed in any suitable way. For example, a user (e.g., user 1916-3) may manually indicate, by way of a GUI of visibility system 1906, that the prescription order has been processed (e.g., the medicine container has been packed) and is ready for delivery. Additionally or alternatively, user 1916-3 may select an option on the GUI of visibility system 1906 to scan, with a camera or scanner included in or connected to user device 1914-3, the printed label on the packed container. In response, visibility system 1906 confirms that the prescription order has been processed and is ready for delivery to patient 1916-5. Illustrative GUIs of visibility system 1906 will be described below.

In method 2000 and method 2100, operations 2002 to 2008 and 2102 trigger a status update (e.g., by the transmission of an event notification 2010 to status system 1910 and/or by performance of operation 2014 by status system 1910). However, prescriber system 1902, prescription management system 1904, visibility system 1906, and delivery system 1908 may each perform multiple different operations, each of which may trigger the status update.

For example, operations that may be performed by prescriber system 1902 that may trigger a status update may include, without limitation, initial creation of a prescription order, approval by the prescriber of a non-formulary drug, receipt of signature by the prescriber, confirmation of the prescription order by a user with approval authority, viewing or modifying a prescription order by a user, sending a reminder notification to a particular care team member to complete a pending step, and transmission of the prescription to prescription management system 1904 or visibility system 1906.

Operations that may be performed by prescription management system 1904 that may trigger a status update may include, without limitation, acknowledgment of receipt of the prescription order from prescriber system 1902, generation of a prescription file, change in approval status of an information module 806 or an information module 1506, submission of a claim to the insurance provider, receipt of notification of insurance claim issues or errors, approval of the insurance claim (e.g., successful billing), final approval of the filled prescription, bagging of the filled prescription, loading of the filled prescription onto a delivery truck, and sending a reminder notification to a particular care team member to complete a pending step. In some examples, a status update may be triggered by any operation that may be logged by prescription management system 1904 or prescription management system 100, including the completion of any of the prescription file queues described above.

Operations that may be performed by visibility system 1906 that may trigger a status update may include, without limitation, acknowledgment of receipt of the prescription order from prescriber system 1902, printing of the received prescription, sending the received prescription to prescription management system 1904 (e.g., by email or fax), receipt of user input indicating that the received prescription has been imported to prescription management system 1904 and/or that processing of the prescription by prescription management system 1904 has begun, receipt of user input indicating that processing of the prescription has been completed (e.g., the prescription has been filled, bagged, and/or loaded onto a delivery truck), scanning of a printed label on the filled prescription container, and receipt of user input indicating any other operation (e.g., receipt of notice of insurance claims issues, approval of insurance claim, etc.).

In examples in which prescription management system 1904 is not communicatively integrated with status system 1910, status system 1910 may set the status of the prescription order as pending pharmacy processing (or some other similar status identifier) in response to receipt of an event notification 2010 from visibility system 1906, such as an event notification 2010 indicating acknowledgment of receipt the prescription order, printing of the prescription order, or commencement of processing of the prescription order by prescription management system 1904. Similarly, status system 1910 may set the status of the prescription order as completed pharmacy processing (or some other similar status identifier) in response to receipt of an event notification 2010 from visibility system 1906, such as an event notification 2010 indicating scanning of the filled prescription container, bagging of the filled prescription container, and/or loading of the filled prescription container onto a delivery truck. In this way, status system 1910 may set the status of a prescription order under processing by prescription management system 1904 even without communicating with prescription management system 1904.

Operations that may be performed by delivery system 1908 that may trigger a status update may include, without limitation, scheduling delivery, loading of the filled prescription container onto the delivery truck, initiation of the delivery route (as may be detected by a tracking system, such as GPS), changes in estimated delivery times (e.g., due to traffic or other delays), a periodic update of the estimated delivery time, a periodic location update of the delivery, and delivery completed.

In some examples, any operation that changes the person or entity responsible for performing a next operation triggers a status change. For example, upon completion of the entry queue in system 100 by a first user (e.g., user 216-1 of user device 204-1), a second user (e.g., user 216-2 of user device 204-2) must review and approve information modules 806 in the entry queue. Accordingly, completion of the entry queue in system 100 may trigger the status update. In some examples, operations that do not change the person or entity responsible for performing a next operation does not trigger the status update. For example, prescription management system 1904 may be configured to require the pack queue and the final queue to be performed by the same person or a person with the same role (e.g., a pharmacist). Accordingly, completion of the pack queue does not trigger the status update.

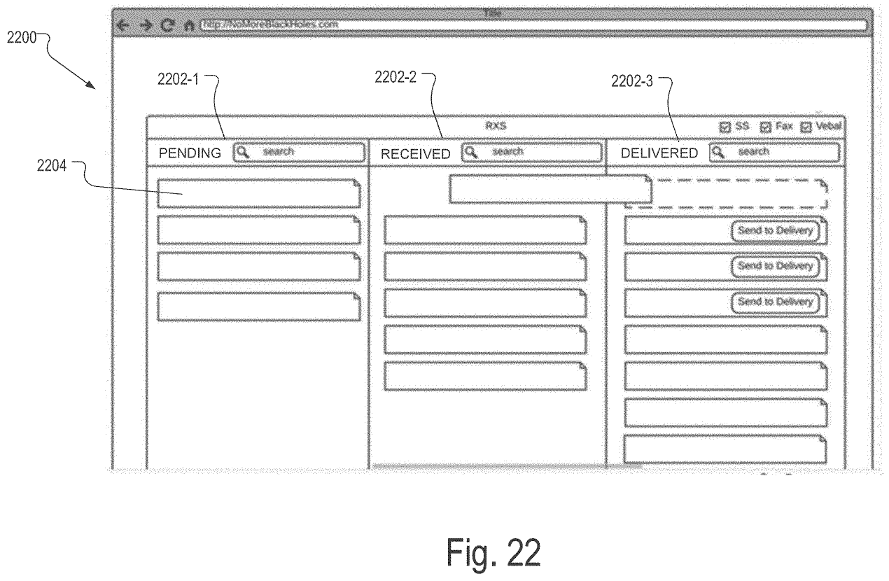

An illustrative example of processing a prescription order and providing a real-time, on-demand status of the prescription order will now be described with reference to FIG. 22. FIG. 22 shows an illustrative dashboard GUI 2200 of visibility system 1906 that may be presented by way of a user device (e.g., user device 1914-3). As visibility system 1906 is associated with pharmacy 1920, dashboard GUI 2200 shows the status of various different prescription order files associated with pharmacy 1920. Dashboard GUI 2200 (“GUI 2200”) includes status regions 2202 each indicating a status of a prescription order file. As shown, GUI 2200 includes a pending status region 2202-1, a received status region 2202-2, and a delivery status region 2202-3. GUI 2200 may include any additional or alternative status regions 2202 as may serve a particular implementation, such as a complete status region. Moreover, status regions 2202 may be arranged in any suitable configuration, whether in side-by-side columns, rows, or any other suitable configuration.

GUI 2200 further includes a prescription order card 2204 (an “Rx card”) for each prescription order and positions each Rx card 2204 within the corresponding status region 2202 based on a current status of the prescription order file (only one Rx card 2204 is labeled in FIG. 22). Although not shown in FIG. 22, Rx cards 2204 may include information identifying the particular prescription order file, such as patient information, prescriber information, and/or order number. In some examples, an Rx card 2204 may be toggled (e.g., in response to a user selecting the Rx card 2204) between a condensed form and an expanded form. In the condensed form, the Rx card 2204 may show a minimal amount of information (e.g., identifying information). In the expanded form, the Rx card 2204 may show additional information, such as the prescribed drug, requested delivery date, more detailed status information (e.g., a most recent logged event), and/or any other suitable information.

Prescriber system 1902 creates a new prescription order based on user input provided by a user (e.g., a nurse, nurse practitioner, or a physician) by way of a new prescription GUI of prescriber system 1902. The new prescription GUI of prescriber system 1902 may include fields for inputting various items of information to request a new prescription order, such as patient name, prescriber name and information, drug information, patient condition, directions, dosage, the pharmacy name and address, and the requested delivery date and time. Upon entering the information, the user may select a selectable submit option to submit the information. When the information is submitted, prescriber system 1902 creates a new prescription order and transmits a new prescription order event notification to status system 1910. Although the new prescription order has been created, it is not yet submitted to the pharmacy, which in this example occurs after the prescription is reviewed and approved by a user having approval authority (e.g., a nurse manager) and signed by a prescriber (e.g., a physician).

Based on the new prescription order event notification, status system 1910 generates a new prescription order file, sets a status of the new prescription order file as pending, and transmits the status of the new prescription order file to prescriber system 1902, prescription management system 1904, visibility system 1906, and/or delivery system 1908, each of which may indicate, at any time upon request by a user, that the status of the prescription order file is “pending” (e.g., pending review, approval, signing, and submission to pharmacy 1920). Thus, in GUI 2200 of visibility system 1906, the Rx card 2204 for the prescription order is positioned within pending status region 2202-1.

A pending prescription order may be approved by a user (e.g., a nurse manager) by way of a GUI presented by prescriber system 1902. For example, user 1916-1 may select an approve button or otherwise provide input by way of user device 1914-1 indicating approval of the pending prescription order. Upon user 1916-1 indicating approval, prescriber system 1902 may report an “approved” status to status system 1910. Status system 1910 may then report this status to any other system. At this time, the status of the prescription order file is still “pending”, so the Rx card 2204 for the prescription order is still positioned within pending status region 2202-1. However, Rx card 2204 may display, in the condensed form and/or the expanded form, the “approved” status and/or a “pending signature” status.

An approved prescription order may be signed by a prescriber (e.g., user 1916-2) by way of a GUI presented by prescriber system 1902 and submitted to the pharmacy (e.g., to prescription management system 1904 or to visibility system 1906 associated with pharmacy 1920) indicated in the prescription. For example, user 1916-2 may select an option on the GUI to submit an e-prescription, an option to submit a voice message prescription, or an option to submit a fax prescription. Upon selecting the option, the user 1916-2 may be presented with an option to sign and submit the prescription to the pharmacy. Upon signing and submitting the prescription, prescriber system 1902 may report a signed and submitted status to status system 1910. Status system 1910 may then report this signed and submitted status to any other system. At this time, the status of the prescription order file may still be indicated as “pending” until prescription management system 1904 (or visibility system 1906) acknowledges receipt, so the Rx card 2204 for the prescription order is still positioned within pending status region 2202-1. However, Rx card 2204 may display, in the condensed form and/or the expanded form, the “signed and sent” status and/or a “pending receipt” status.

In other examples, status system 1910 may update the status of the prescription order to “received” when the prescription order is signed and sent, even without acknowledgment of receipt of the prescription order by prescription management system 1904 or visibility system 1906. In these examples, the Rx card 2204 for the prescription order may be positioned within received status region 2202-2. However, Rx card 2204 may display, in the condensed form and/or the expanded form, the “signed and sent” status and/or a “pending receipt acknowledgment” status.

In some examples, the order of performing the approval and the sign/submit steps may be switched. In further examples, the approval and sign/submit functionality may be restricted to certain users (e.g., certain user IDs) or users with a certain role (e.g., nurse practitioner, nurse manager, physician, etc.), thereby ensuring that a prescription is not submitted to the pharmacy prior to all required steps being completed first. This may help prevent black hole delays caused by incomplete or incorrect prescriptions being submitted to the pharmacy.

When the prescription order is received by prescription management system 1904 or visibility system 1906, prescription management system 1904 or visibility system 1906 may automatically acknowledge receipt of the prescription. Alternatively, a user (e.g., user 1916-3) may be required to manually acknowledge receipt of the prescription order. Upon acceptance of the prescription order, prescription management system 1904 or visibility system 1906 may report a receipt acknowledgment to status system 1910. Status system 1910 may then report this status to any other system. At this time, status system 1910 may move the Rx card 2204 for the prescription order from pending status region 2202-1 to received status region 2202-2.

In some examples, prescription management system 1904 is communicatively integrated with visibility system 1906 so that prescription orders pass through both systems and can be automatically tracked as the prescription is processed and filled. For example, if visibility system 1906 first receives and acknowledges receipt of the prescription order, visibility system 1906 may forward the prescription order to prescription management system 1904 for processing.

After prescription management system 1904 receives the prescription order, the prescription is processed by using prescription management system 1904. The prescription may be processed in any suitable way, including in any way described herein. During processing by prescription management system 1904, the Rx card 2204 for the prescription order may be positioned within received status region 2202-2. In some examples, various processing operations may be reported to status system 1910, which may provide various processing status updates that may be presented within Rx card 2204 (e.g., in an expanded form of Rx card 2204).

When prescription management system 1904 confirms that the prescribed medicine has been filled, prescription management system 1904 may send the prescription file to delivery system 1908 for delivery processing (e.g., scheduling, estimating a delivery time, identifying a delivery route, etc.). Prescription management system 1904 may confirm that the prescribed medicine has been filled based on operations performed by prescription management system 1904, such as printing the label for the container or bagging the container and/or scheduling the delivery in the delivery queue of prescription management system 1904. Prescription management system 1904 reports the filled/completed status to status system 1910. Status system 1910 may then report the filled status to any other system. At this time, status system 1910 may move the Rx card 2204 for the prescription order from received status region 2202-2 to delivery status region 2202-3.

In some examples, prescription management system 1904 may not communicatively integrate with status system 1910, visibility system 1906, and delivery system 1908. Accordingly, the prescription order may be advanced from visibility system 1906 to delivery system 1908, and the status updated accordingly, based on some other operation that is not performed by prescription management system 1904. For example, when a prescription order is received by visibility system 1906 and then printed so that the prescription information can be scanned or entered into prescription management system 1904, visibility system 1906 may report the printing to status system 1910. Status system 1910 may then report a delivery pending status to any other system. At this time, status system 1910 may move the Rx card 2204 for the prescription order from received status region 2202-2 to delivery status region 2202-3.

Alternatively to updating the status based on printing the prescription order received at visibility system 1906, a user may interact with GUI 2200 to provide user input to move Rx card 2204 to delivery status region 2202-3. For example, a user may select Rx card 2204 in the received status region 2202-2 and drag Rx card 2204 card to delivery status region 2202-3. As another example, the user may select a button or other option on Rx card 2204 and select, from a drop-down menu, the delivery status region 2202-3 to which Rx card 2204 is to be moved. Upon manually moving Rx card 2204 from received status region 2202-2 to delivery status region 2202-3, visibility system 1906 and/or delivery system 1908 may report the changed status to status system 1910. Status system 1910 may then report a delivery pending status to any other system. At this time, the Rx card 2204 for the prescription order remains in the delivery status region 2202-3.

Upon delivery of the filled prescription to the patient, delivery system 1908 may confirm that the filled prescription was delivered to the patient and mark the delivery as complete. Delivery system 1908 may confirm that the filled prescription was delivered in any suitable way. For example, a delivery driver (e.g., user 1916-4) may provide user input, by way of GUI 2200, to indicate that the medication was delivered. For instance, the user may drag and drop Rx card 2204 from the delivery status region 2202-3 to a completed status region. Alternatively, the user may select a “delivery complete” selectable option on the Rx card 2204. In further examples, delivery system 1908 may automatically indicate completion of delivery based on a GPS location of the delivery driver matching the delivery location indicated on the prescription or based on a confirmation provided by the patient by way of the patient's user device. Upon confirming delivery of the filled prescription, delivery system 1908 may report the delivered status to status system 1910. Status system 1910 may then report a delivery completed status to any other system. At this time, system 1910 may move the Rx card 2204 from the delivery status region 2202-3 to a completed status region. If GUI 2200 does not have a completed status region, system 1910 may remove the Rx card 2204 from GUI 2200.

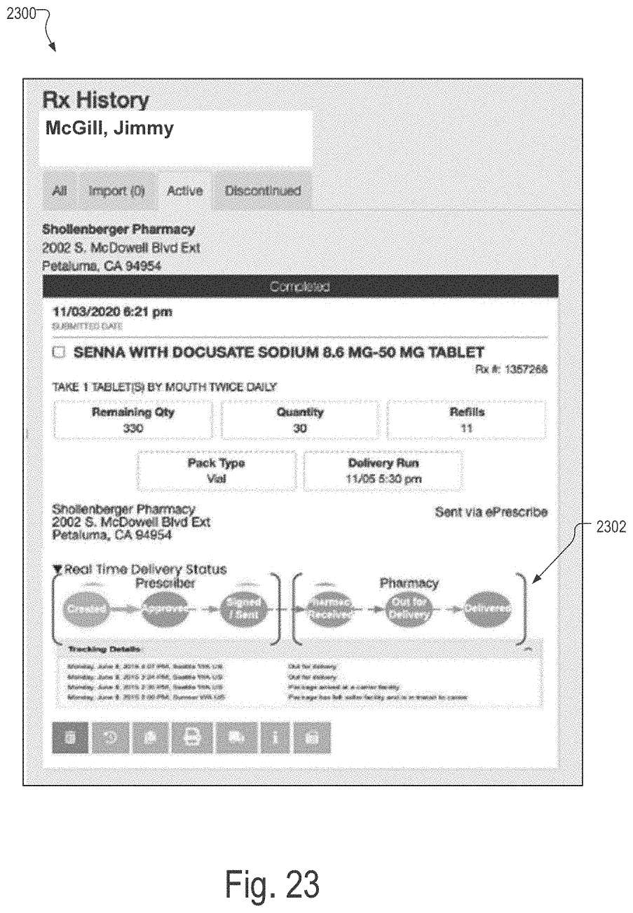

In the examples just described, GUI 2200 of FIG. 22 shows a dashboard indicating the status of multiple different prescription orders. FIG. 23 shows an illustrative GUI 2300 that shows a status of a selected prescription order. GUI 2300 may be presented by prescriber system 1902, prescription management system 1904, visibility system 1906, and/or delivery system 1908. For example, GUI 2300 may be presented by prescriber system 1902 upon selecting a specific patient file within a GUI provided by prescriber system 1902. In other examples, GUI 2300 may be presented by prescription management system 1904 upon selection of a particular prescription file. For instance, GUI 2300 may be presented in document window 306 or action window 308 upon selection of a document identifier 312 or a prescription file identifier 402. In further examples, GUI 2300 may be presented by visibility system 1906 and/or delivery system 1908 upon selection of an Rx card 2204 in GUI 2200. GUI 2300 may be presented as a popup window superimposed over a GUI (e.g., GUI 300 or GUI 2200) or as an expanded form of Rx card 2204. In yet further examples, GUI 2300 may be presented on a user device (e.g., user device 1914-5) associated with a patient (user 1916-5) or a family caregiver.

As shown in FIG. 23, GUI 2300 includes, in addition to prescription order information (e.g., patient information, drug information, prescriber information, etc.), a status indicator 2302 that shows a status of the prescription identified in GUI 2300. Status indicator 2302 may have any suitable form. In the example of FIG. 23, status indicator 2302 includes a series of graphical elements (e.g., icons, text bubble, etc.) each representing various stages of the prescription process (e.g., created, approved, signed/sent, received at pharmacy, out for delivery, delivered). The graphical element of a current status is identified, such as by altering an appearance (e.g., a color, a shape, a size, and/or text) of the graphical element of the current status. In some examples, status indicator 2302 also indicates which entity and/or care team member is responsible for completing the current task.

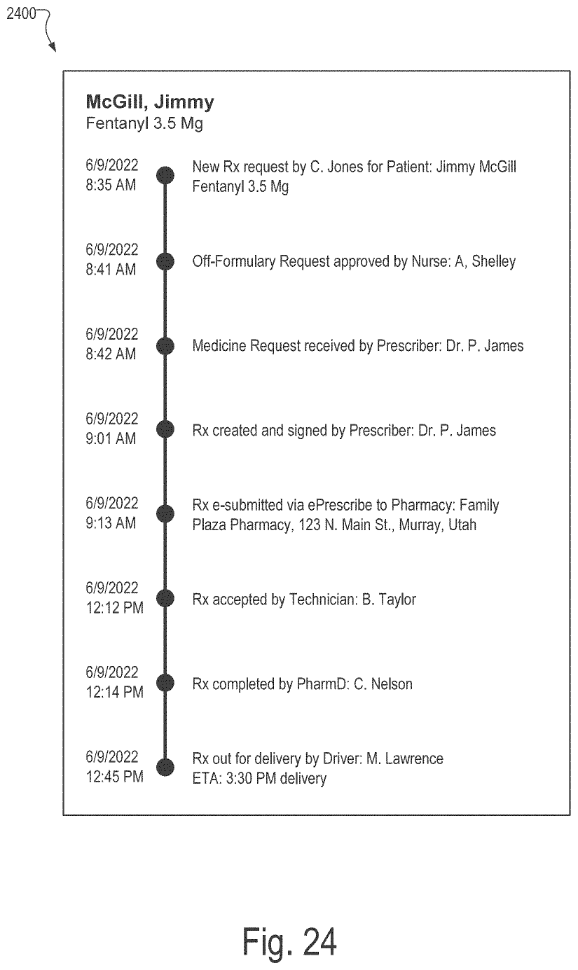

In some examples, a user may select status indicator 2302 to access a more detailed status report for the associated prescription order. FIG. 24 shows a detailed status report 2400 that may be accessed by selecting status indicator 2302. Status report 2400 may be presented by way of a GUI of prescriber system 1902, prescription management system 1904, visibility system 1906, and/or delivery system 1908. Status report 2400 may present a list of events logged by status system 1910 and/or other related information, such as an estimated delivery time for the delivery driver, and a current location of the delivery driver.

Status indicator 2302 and status report 2400 may be presented in any GUI as may suit a particular implementation. For example, status indicator 2302 may be presented on Rx card 2204. In further examples, status indicator 2302 and/or status report 2400 may be delivered to a user device 1914 by way of a message, such as a text message, a multimedia message, an email message, or an instant message from status system 1910.

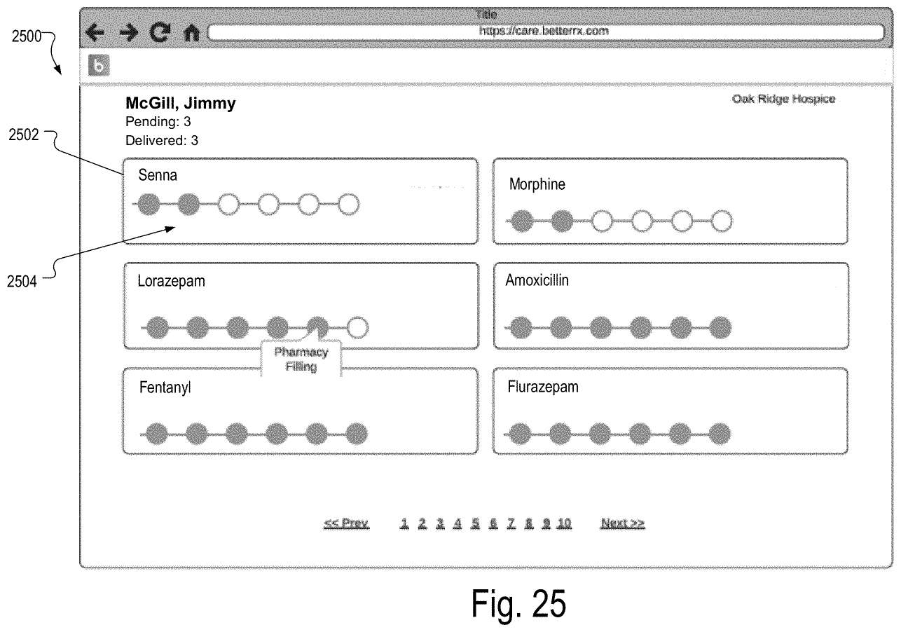

FIG. 25 shows another illustrative GUI 2500 that may be viewed by a care team member (e.g., nurse 1916-1, physician 1916-2, pharmacist 1916-3, or patient 1916-5). GUI 2500 may be provided by any one or more of prescriber system 1902, prescription management system 1904, and visibility system 1906. As shown, GUI 2500 displays an Rx card 2502 for each medication prescribed for a particular patient. Each Rx card includes a status indicator 2504 indicating the status of the prescribed medication. Status indicator 2504 may have any suitable format, including any format shown or described herein. Status indicator 2504 may be selected to view detailed status information (e.g., status report 2400) and or to perform certain operations, such as message another care team member or request a reminder notification be sent to the care team member or facility responsible for completing the currently pending task. GUI 2500 may have any other format and configuration as may suit a particular implementation. For example, prescription orders and their corresponding status indicators 2504 may be presented in a table format rather than in Rx cards 2502.

In some examples, a guest (e.g., a patient 1916-5, a family member of patient 1916-5, a person with a power of attorney, etc.) may be provided with access to view the current status of the prescription order. To this end, a member of the care team (e.g., a nurse) may invite the guest to sign up for access to view the prescription status. Access may be given through a guest access system, which may be any one of prescriber system 1902, prescription management system 1904, visibility system 1906, delivery system 1908, or status system 1910. The invitation may apply to a particular prescription or may be a one-time sign-up for all prescriptions for the patient. The invitation may be sent to the guest in any suitable way, such as by text message, email, voice message, or through an application executed by the user device 1914-5.

The invitation message may provide the guest with a verification test. Any suitable verification test may be used. For example, the guest's mobile device (e.g., user device 1914-5) may receive the text message with a link to a verification webpage by which the user may provide verification information, such as patient name, date of birth, doctor, and/or the guest's information (if different from the patient). If the verification information matches information stored with the guest access system, the guest access system may then set up an account for the guest or otherwise provide the guest with access to real-time, on-demand prescription status information. For example, the guest access system may present, by way of a dedicated application or a browser on the guest's user device, GUI 2200 (limited to prescription orders for the patient), GUI 2300, and/or any other GUI with status indicator 2302 and/or status report 2400.

Access to view a status of a prescription may be managed by a care team member through the guest access system. For example, access may be revoked and/or modified.

To further facilitate timely provisioning of medication to a patient, status system 1910 (and/or any of systems 1902 to 1908) may be configured to provide automatic notifications to a user, such as when the status of a prescription has changed or when a particular status has remained unchanged for a threshold period of time. The notification may be sent to a specific care team member or a defined set of care team members, or to all care team members. For example, when a prescription has been approved by a nurse manager, status system 1910 and/or prescriber system 1902 may send a notification to the physician (e.g., to the physician's user device 1914-2) indicating that the physician's signature on the prescription is required. If the status of the prescription remains as “need signature” for more than a threshold time period (e.g., 2 hours), another notification or reminder may be sent to the physician and/or nurse or nurse manager. In this way, processing of a prescription may be kept on track without falling into a black hole. In some examples, a user may set user settings to configure notification reminders, such as the period of time to elapse before sending each reminder for each status, where to send the notification, notification contents, notification format (e.g., text message, email, audible alarm, etc.), and the like. In some examples, the notification is provided by a GUI of the respective system through the intended recipient's account. For instance, a GUI of prescriber system 1902 may include a messages feature and/or may provide a notification by way of a popup on the GUI.

With platform 1900, the real-time status of a prescription may be viewed at any time by any member of the care team, as well as by the patient or a guest of the patient. Since each team member may have visibility to the prescription status, black holes that cause delays can be avoided and, if they do occur, quickly remedied.

In platform 1900 described herein, systems 1902 to 1908 report status updates to status system 1910, which maintains a database of prescription files and logs status reports for each prescription file. Thus, the GUI of each system may provide a status indicator indicating the current status of the prescription. The status information used to generate the status indicator is pulled (or pushed) from status system 1910. The status can be displayed on individual orders (e.g., patient cards, prescription cards, medication cards) and/or on a dashboard that provides an overall view of multiple different prescriptions and/or patients. Any user at any device has visibility to information about the status of the medication. Thus, platform 1900 uniquely provides end-to-end visibility, from prescriber to pharmacy to delivery to patient, and accountability for the care team (nurse, physician/prescriber, pharmacy).

Since platform 1900 provides end-to-end tracking of medication provisioning, platform 1900 also enables tracking the timing for medication provisioning. For example, any one or more of systems 1902 to 1910 may track the amount of time it takes to complete various tasks and stages within the medication provisioning process, and how long each user takes to complete a required step. This information may be used to speed up the medication provisioning process, such as by timing the delivery of reminder notifications, providing scores or reports to users, optimal scheduling of deliveries, and estimating delivery times.

In some examples, a prescriber computing device (e.g., user device 1914-1 or user device 1914-2) may execute an application to interact with one or more features provided by prescriber system 1902. The application may be a browser-based application, or it may be a standalone application installed on the mobile computing device. For example, prescriber system 1902 may be used to electronically upload patient and prescription documents, input patient and prescription information, receive notification that the prescription has been approved by the pharmacist, address clinical review issues identified during a prescription review, receive notification of and/or address insurance billing issues, to receive notification that the prescription has been picked up or delivered, view the current status of a prescription, and/or to securely communicate with the pharmacy.

In some examples, a pharmacy computing device (e.g., user device 1914-3) may execute an application to interact with one or more features provided by prescription management system 1904 and/or visibility system 1906. The application may be a browser-based application, or it may be a standalone application installed on the mobile computing device. For example, prescription management system 1904 may be used to receive, process, and fill prescriptions, manage medication inventory, manage patient and customer records, and communicate with insurance.

In some examples, a mobile computing device (e.g., a mobile phone, a table computer, a laptop computer, etc.) may execute an application to interact with one or more features provided by delivery system 1908. The application may be a browser-based application, or it may be a standalone application installed on the mobile computing device. Thus, a delivery driver (e.g., user 1916-4) may use the mobile computing device (e.g., user device 1914-4) in the delivery of medications to patients, such as to view routes, schedules, and to confirm successful delivery of medications. For example, the delivery user may scan (e.g., with a dedicated scanner connected to the mobile computing device or a camera in the mobile computing device) the labels on the medicine bottles when the medicine is loaded onto the delivery truck and/or when the medicine is delivered to the patient.

In some examples, a patient computing device (e.g., a mobile phone, a tablet computer, a personal computer, etc.) may also execute an application to view a status of a prescription or communicate with a care team member. The application may be a browser-based application, or it may be a standalone application installed on the mobile computing device. For example, patient device 1914-5 may be used by patient 1916-5 to request a new prescription or refill a prescription, electronically upload patient and prescription documents (e.g., scan paper prescriptions, health charts, etc.), receive notification of and/or address insurance billing issues, review pricing information (e.g., estimated costs for the medicine, costs of alternative or generic medications), pay for medication, receive notification that the prescription is available for pick up, track delivery of the prescription, securely communicate with the pharmacist, review directions for taking the medication, view drug information (e.g., side effects, etc.), view photos of the drugs, and the like.

In some examples, any of systems 1902 to 1910 may provide location-, user-, and/or role-based rights permissions for any one or more features provided by systems 1902 to 1910. The rights permissions may be in any suitable form, such as read-only access or no access (e.g., do not appear on the GUI). For example, delivery driver 1916-4 may be restricted from viewing status information for prescriptions and medications, patient health conditions, etc.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

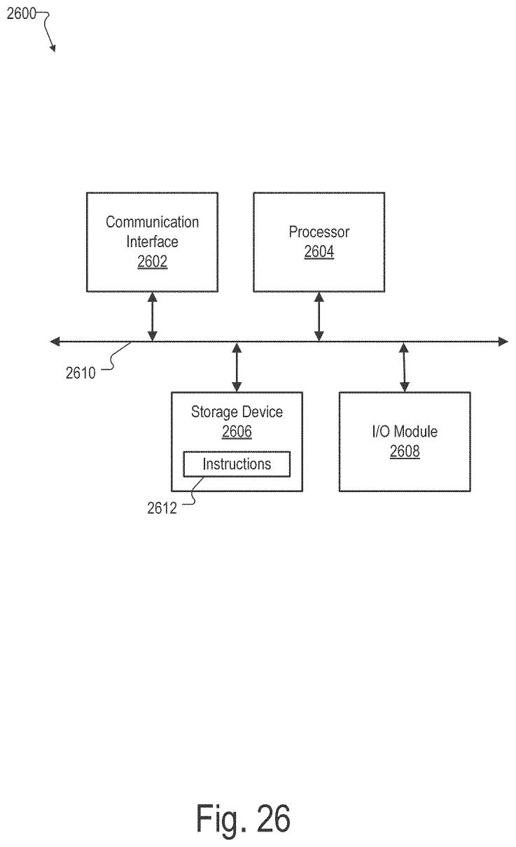

FIG. 26 illustrates an exemplary computing device 2600 that may be specifically configured to perform one or more of the processes described herein. Any of the systems, units, computing devices, and/or other components described herein may be implemented by computing device 2600.

As shown in FIG. 26, computing device 2600 may include a communication interface 2602, a processor 2604, a storage device 2606, and an input/output (“I/O”) module 2608 communicatively connected one to another via a communication infrastructure 2610. While an exemplary computing device 2600 is shown in FIG. 26, the components illustrated in FIG. 26 are not intended to be limiting. Additional or alternative components may be used in other embodiments. Components of computing device 2600 shown in FIG. 26 will now be described in additional detail.

Communication interface 2602 may be configured to communicate with one or more computing devices. Examples of communication interface 2602 include, without limitation, a wired network interface (such as a network interface card), a wireless network interface (such as a wireless network interface card), a modem, an audio/video connection, and any other suitable interface.

Processor 2604 generally represents any type or form of processing unit capable of processing data and/or interpreting, executing, and/or directing execution of one or more of the instructions, processes, and/or operations described herein. Processor 2604 may perform operations by executing computer-executable instructions 2612 (e.g., an application, software, code, and/or other executable data instance) stored in storage device 2606.

Storage device 2606 may include one or more data storage media, devices, or configurations and may employ any type, form, and combination of data storage media and/or device. For example, storage device 2606 may include, but is not limited to, any combination of the non-volatile media and/or volatile media described herein. Electronic data, including data described herein, may be temporarily and/or permanently stored in storage device 2606. For example, data representative of computer-executable instructions 2612 configured to direct processor 2604 to perform any of the operations described herein may be stored within storage device 2606. In some examples, data may be arranged in one or more databases residing within storage device 2606.

I/O module 2608 may include one or more I/O modules configured to receive user input and provide user output. I/O module 2608 may include any hardware, firmware, software, or combination thereof supportive of input and output capabilities. For example, I/O module 2608 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2608 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2608 is configured to provide graphical data to a display for presentation to a user. The graphical data may be representative of one or more graphical user interfaces and/or any other graphical content as may serve a particular implementation.

In the preceding description, various illustrative embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1-4. (canceled)

5. A status reporter system comprising:
a memory storing instructions; and
a processor communicatively coupled to the memory and configured to execute the instructions to:
receive, based on a trigger event that occurs with respect to a prescription order at one of a prescriber system associated with a medical provider, a prescription management system associated with a pharmacy, and a delivery system associated with the pharmacy, an event notification that identifies the prescription order;
identify, based on the event notification, a type of the trigger event;
set, based on the type of the trigger event, a status of the prescription order; and
provide, for display within a graphical user interface of a visibility system, an indication of the status of the prescription order.

6. The status reporter system of claim 5, wherein:
the trigger event comprises creation of the prescription order at the prescriber system;
the event notification is received from the prescriber system;
the prescriber system is communicatively coupled with the prescription management system and is configured to:
receive user input approving the prescription order; and
transmit, based on receipt of the user input approving the prescription order, the prescription order to the prescription management system; and
the providing the indication of the status of the prescription order is performed before the prescription management system receives the prescription order from the prescriber system.

7. The status reporter system of claim 5, wherein the type of the trigger event is identified based on a current status of the prescription order and one or more of an identity of the one of the prescriber system, the prescription management system, and the delivery system.

8. The status reporter system of claim 5, wherein:
the graphical user interface of the visibility system comprises:
a first status region comprising one or more Rx cards each identifying a prescription order having a first status;
a second status region comprising one or more Rx cards each identifying a prescription order having a second status that is different than the first status;
a third status region comprising one or more Rx cards each identifying a prescription order having a third status that is different than the first status and the second status; and
the providing the indication of the status of the prescription order comprises providing an Rx card identifying the prescription order within one of the first status region, the second status region, and the third status region based on the status of the prescription order.

9. The status reporter system of claim 8, wherein:
the first status indicates that a respective prescription order is pending transmission to the prescription management system;
the second status indicates that the respective prescription order has been received by the prescription management system or the visibility system; and
the third status indicates that prescription processing of the respective prescription order has been completed by way of the prescription management system.

10. The status reporter system of claim 5, wherein:
the trigger event comprises a determination that a prescription identified in the prescription order has been picked-up or delivered to a delivery location identified in the prescription order;
the event notification is received from the delivery system; and
the processor is further configured to execute the instructions to provide, for display within a graphical user interface of the prescriber system, an additional indication of the status of the prescription order.

11. The status reporter system of claim 5, wherein:
the graphical user interface of the visibility system comprises a status report listing a plurality of trigger events that have occurred with respect to the prescription order; and
the providing the indication of the status of the prescription order comprises adding the trigger event to the status report.

12. The status reporter system of claim 5, wherein the processor is further configured to:
determine that the status of the prescription order has remained unchanged for a threshold period of time; and
provide, based on the determination that the status of the prescription order has remained unchanged for the threshold period of time, a reminder notification to a user device associated with the prescriber system, the prescription mana.gement system, the delivery system, or the visibility system.

13. The status reporter system of claim 5, wherein the processor is further configured to provide, in response to the setting the status of the prescription order, a notification to a user device associated with the prescriber system, the prescription management system, the delivery system or the visibility system, the notification indicating that the status of the prescription order has changed.

14. A method comprising:
receiving, by a status reporter system based on a trigger event that occurs with respect to a prescription order at one of a prescriber system associated with a medical provider, a prescription management system associated with a pharmacy, and a delivery system associated with the phartrtacy, an event notification that identifies the prescription order;
identifying, based on the event notification, a type of the trigger event;
setting, based on the type of the trigger event, a status of the prescription order; and
providing, for display within a graphical user interface of a visibility system, an indication of the status of the prescription order.

15. The method of claim 14, wherein:
the trigger event comprises creation of the prescription order at the prescriber system;
the event notification is received from the prescriber system;
the prescriber system is communicatively coupled with the prescription management system and is configured to:
receive user input approving the prescription order; and
transmit, based on receipt of the user input approving the prescription order, the prescription order to the prescription management system; and
the providing the indication of the status of the prescription order is performed before the prescription management system receives the prescription order from the prescriber system.

16. The method of claim 14, wherein the type of the trigger event is identified based on a current status of the prescription order and one or more of an identity of the one of the prescriber system, the prescription management system, and the delivery system.

17. The method of claim 14, wherein:
the graphical user interface of the visibility system comprises:
a first status region comprising one or more Rx cards each identifying a prescription order having a first status;
a second status region comprising one or more Rx cards each identifying a prescription order having a second status that is different than the first status;
a third status region comprising one or more Rx cards each identifying a prescription order having a third status that is different than the first status and the second status; and
the providing the indication of the status of the prescription order comprises providing an Rx card identifying the prescription order within one of the first status region, the second status region, and the third status region based on the status of the prescription order.

18. The method of claim 14, wherein:
the trigger event comprises a determination that a prescription identified in the prescription order has been picked-up or delivered to a delivery location identified in the prescription order;
the event notification is received from the delivery system; and
the method further comprises providing, for display within a graphical user interface of the prescriber system, an additional indication of the status of the prescription order.

19. The method of claim 14, wherein:
the graphical user interface of the visibility system comprises a status report listing a plurality of trigger events that have occurred with respect to the prescription order; and
the providing the indication of the status of the prescription order comprises adding the trigger event to the status report.

20. The method of claim 14, further comprising:
determining that the status of the prescription order has remained unchanged for a threshold period of time, and
providing, based on the determination that the status of the prescription order has remained unchanged for the threshold period of time, a reminder notification to a user device associated with the prescriber system, the prescription management system, the delivery system, or the visibility system.

21. The method of claim 14, further comprising:
providing, in response to the setting the status of the prescription order, a notification to a user device associated with the prescriber system, the prescription management system, the delivery system, or the visibility system, the notification indicating that, the status of the prescription order has changed.

22. A status reporter system comprising:
a memory storing instructions; and
a processor communicatively coupled to the memory and configured to execute the instructions to:
receive, based on each trigger event that occurs with respect to a prescription order at each of a prescriber systei i associated with a medical provider, a prescription management system associated with a pharmacy, and a delivery system associated with the pharmacy, an event notification that identifies the prescription order;
log, based on each event notification, each trigger event that occurs with respect to the prescription order; and
provide, for display within a graphical user interface of a visibility system, a status report that lists each of the logged trigger events that occurs with respect, to the prescription order.

23. The status reporter system of claim 22, wherein:
each event notification further comprises a timestamp of the respective trigger event; and
the status report further lists the timestamp corresponding to each logged trigger event.

24. The status reporter system of claim 22, wherein:
the graphical user interface of the visibility system comprises a selectable Rx card associated with the prescription order; and
the processor is configured to execute the instructions to provide the status report in response to a user selection of the Rx card associated with the prescription order.
